(19) United States
(12) Patent Application Publication
(10) Pub. No.: US 2015/0057608 A1
(43) Pub. Date: Feb. 26, 2015

(54) Delivery Device and Components Thereof

(71) Applicant: Biogen Idec MA Inc., Cambridge, MA (US)

(72) Inventors: Carl F. Hitscherich, JR., Marblehead, MA (US); Soham Bhatt, Cambridge, MA (US); Nam Chen, Cambridge, MA (US); Chase Fetzer, Shirley, MA (US)

(21) Appl. No.: 14/387,939

(22) PCT Filed: Mar. 28, 2013
(86) PCT No.: PCT/US13/34396

§ 371 Date: Sep. 25, 2014

Related U.S. Application Data
(60) Provisional application No. 61/617,340, filed on Mar. 29, 2012.

(51) Int. Cl. A61M 5/31; A61M 5/19; A61M 5/00; A61M 5/315

(52) U.S. Cl. CPC A61M 5/3137; A61M 5/31511; A61M 5/19; A61M 5/002; A61M 2205/584; A61M 2209/06; A61M 2202/0452; A61M 2005/31516; A61M 2005/1787
USPC 604/91; 604/227; 604/218; 604/189

(57) ABSTRACT

Delivery devices and components thereof are disclosed. In an embodiment, a delivery device may include a barrel holding a liquid for delivery. The delivery device may also include a finger flange assembly coupled to the barrel. The finger flange assembly may include a first flange and a second flange each extending laterally from opposite sides of the finger flange assembly, wherein the first flange and the second flange each curve toward the barrel. The delivery device may also include a plunger coupled to the barrel and passing through the finger flange assembly. The plunger may also include a disc-shaped head.

### FIELD OF THE INVENTION

The present invention generally relates to delivery devices and components thereof. More particularly, embodiments of the present invention relate to medical syringes and components thereof having improved ergonomics, versatility, and/or ease of use.

### BACKGROUND OF THE INVENTION

Delivery devices, such as syringes, are generally used as pumps to take in and/or expel materials. Delivery devices may be used for a wide variety of medical and non-medical purposes. Medical uses for delivery devices, such as syringes, may include, for example, administering a therapeutic injection to a patient or withdrawing blood from a patient. Non-medical uses for delivery devices may include, for example, applying an adhesive compound while constructing an article of manufacture or injecting a flavored liquid into a food product while cooking. While delivery devices, such as syringes, have been around in one form or another for centuries, there still remains a need for new delivery devices that are capable of offering improved ergonomics, versatility, and/or ease of use.

Many medical conditions and disorders require the administration of a medicament via a syringe. Such conditions and disorders include conditions or disorders of coagulation, or blood clotting. Coagulation is a complex process by which blood forms clots. It is an important part of hemostasis, the cessation of blood loss from a damaged vessel, wherein a damaged blood vessel wall is covered by a platelet and fibrin-containing clot to stop bleeding and begin repair of the damaged vessel. Conditions and disorders of coagulation can lead to an increased risk of bleeding (hemorrhage) or obstructive clotting (thrombosis).

Hemophilia is one of the most common inherited coagulation disorders in the world. It results in decreased in vivo and in vitro blood clotting activity, and requires extensive medical monitoring throughout the life of the affected individual. In the absence of intervention, the individual affected by will suffer from spontaneous bleeding in the joints, muscles, throat, neck, kidneys, and other parts of the body, often leading to other serious medical complications. Severe bleeding may also result from routine surgery, dental extractions, and even minor injuries. Even microbleeds—pinpoint drops of blood that leak from blood vessels—may cause severe damage to the body over time.

In hemophilia, coagulation is disturbed by a lack of certain plasma blood clotting factors. For example, hemophilia A is caused by a deficiency in Factor VIII (FVIII), while hemophilia B is caused by a deficiency in Factor IX (FIX). Each of these forms of hemophilia may result from either the decreased synthesis of the relevant blood clotting factor protein (e.g. FVIII or FIX) or a defective blood clotting factor protein with reduced activity.

The treatment of hemophilia occurs by replacing the missing blood clotting factor protein by exogenous factor concentrates highly enriched in the missing clotting factor. However, generating such a concentrate from blood is fraught with technical difficulties. In addition, replacement clotting factors typically have short half-lives and therefore require frequent dosing for affected individuals.

The necessary blood clotting factor proteins are typically administered at least several times a week via intravenous injections to the individual affected by hemophilia using a syringe. While some affected individuals may receive injections from a caregiver, many individuals chose to self-administer injections, often for reasons of cost and convenience.

Because individuals affected by hemophilia are highly susceptible to accidental bleeding in everyday situations, stress points on tools (e.g. syringes), stress points generated by environmental surfaces (e.g. the surface or edge of a table), and stresses resulting from unnatural movements of the body have the potential to cause microbleeds as the individual interacts with tools in their environment. In the case of routine injections using a syringe, particularly for affected individuals who self-administer treatments, even small positive changes in syringe configurations and the movements necessary to administer and receive an injection have the potential to minimize microbleeds or other injuries to the individual.

Reduced mortality, prevention of joint damage, and improved quality of life have been important achievements resulting from the development of suitable replacement blood clotting factors, such as FVIII and FIX. However, currently available syringes for administering these treatments have many structural and functional shortcomings that make them suboptimal—and in some cases even potentially harmful—to individuals affected by hemophilia.

Therefore, there remains a need for new delivery devices, such as syringes, offering improved ergonomics, versatility, and/or ease of use, particularly for use by patients or caregivers administering hemophilia treatments.

It should be noted, however, that while the present discussion focuses primarily on the use of syringes for medical purposes generally, and for the treatment of hemophilia specifically, the embodiments of delivery devices described herein need not be limited to syringes, need not be so limited in application, and may be suitable for a variety of non-medical purposes, or for treating medical conditions beyond hemophilia.

### BRIEF SUMMARY OF THE INVENTION

Embodiments of the present invention relate to a delivery device, such as a syringe. The delivery device may include a barrel holding a liquid for delivery. The delivery device may also include a finger flange assembly coupled to the barrel. The finger flange assembly may include a first flange and a second flange each extending laterally from opposite sides of the finger flange assembly, wherein the first flange and the second flange each curve toward the barrel. The delivery device may also include a plunger coupled to the barrel and passing through the finger flange assembly. The plunger may include a disc-shaped head.

Embodiments of the present invention also relate to a finger flange assembly configured for use with a delivery device, such as a syringe. The finger flange assembly may include a substantially cylindrical trunk having a proximal end and a distal end, wherein the distal end of the trunk is configured to attach to the barrel of the delivery device. The finger flange assembly may also include a first flange and a second flange each extending laterally from opposite sides of the proximal end of the trunk, wherein the first flange and the second flange each curve toward the distal direction and terminate in respective first and second flange tips, wherein the first flange and the second flange each become narrower as they extend toward the respective first and second flange tips.

Embodiments of the present invention further relate to a plunger configured for use with a delivery device, such as a syringe. The plunger may include a circular disc-shaped head at a proximal end of the plunger. The plunger may also include a rod coupled to and extending distally from the head, wherein the proximal surface of the head includes a spherical dimple.

In another aspect, embodiments of the present invention are directed to a kit comprising the delivery device described herein and a package insert. The package insert can include an instruction for using the delivery device, for administering a medicament, or for treating the disorder or condition.

Embodiments of the delivery devices described herein can include a medicament, which may be any therapeutic that can be administered in a liquid formulation. In some embodiments, medicaments may include small molecule biologics, large molecule biologics, and proteins. In one embodiment, the medicament may be a protein capable of treating a blood clotting disorder, such as a clotting factor. The protein may be lyophilized.

In one embodiment, the protein is a FVIII protein. In another embodiment, the protein is a FIX protein. The FVIII protein can be any FVIII known in the art such as, for example, a functional fragment, a variant, an analog, or a derivative thereof that retains the function of full-length wild-type FVIII in the coagulation pathway. The FVIII protein can be the human, porcine, canine, rat, or murine FVIII protein. In other embodiments, the FVIII or FIX protein is a chimeric protein. The chimeric protein can comprise a FVIII or FIX subsequence and a second substance. In one embodiment, the second subsequence is capable of extending the half-life of the FVIII protein. In another embodiment, the second substance comprises an Fc or an albumin. In other embodiment, the FVIII or FIX protein can further comprise an additional subsequence, such as an additional Fc.

Further embodiments, features, and advantages of the present invention, as well as the structure and operation of the various embodiments of the present invention, are described in detail below with reference to the accompanying drawings.

### BRIEF DESCRIPTION OF THE FIGURES

The accompanying drawings, which are incorporated herein and form a part of the specification, illustrate the present invention by way of example, and not by way of limitation, and, together with the description, further serve to explain the principles of the invention and to enable a person skilled in the pertinent art to make and use the invention.

FIG. 1 is a front view of a delivery device according to an embodiment of the present invention.

FIG. 2 is a front perspective view from above of a delivery device according to an embodiment of the present invention.

FIG. 3 is a side view of a delivery device barrel according to an embodiment of the present invention.

FIG. 4 is a close-up front view of a portion of a delivery device barrel according to an embodiment of the present invention.

FIG. 5A is front perspective view of a delivery device barrel hub according to an embodiment of the present invention.

FIG. 5B is front perspective view of a delivery device barrel hub according to an embodiment of the present invention.

FIG. 6 is an illustration of an infusion set according to an embodiment of the present invention.

FIG. 7A is a front view of a finger flange assembly according to an embodiment of the present invention.

FIG. 7B is a front perspective view from above of a finger flange assembly according to an embodiment of the present invention.

FIG. 8A is a top view of a finger flange assembly according to an embodiment of the present invention.

FIG. 8B is an isometric view of a finger flange assembly according to an embodiment of the present invention.

FIG. 9 is a side view of a finger flange assembly according to an embodiment of the present invention.

FIGS. 10A-10D are various schematic views of a finger flange assembly according to an embodiment of the present invention.

FIG. 11A is a top view of a plunger head according to an embodiment of the present invention.

FIG. 11B is a front perspective view from above of a plunger head according to an embodiment of the present invention.

FIG. 11C is a front perspective view from below of a plunger head according to an embodiment of the present invention.

FIG. 12A is a side view of a plunger according to an embodiment of the present invention.

FIG. 12B is a front view of a plunger according to an embodiment of the present invention.

FIG. 12C is a sectional view of the plunger of FIG. 12B taken along the line A-A according to an embodiment of the present invention.

FIG. 13 is a close-up sectional view of a portion of the plunger of FIG. 12B taken along the line A-A according to an embodiment of the present invention.

FIGS. 14A-14D are various schematic views of a plunger according to an embodiment of the present invention.

FIG. 15A is an illustration of a person's hand holding a delivery device according to an embodiment of the present invention.

FIG. 15B is an illustration of a person's hand holding a delivery device according to an embodiment of the present invention.

FIG. 16 is an illustration of a person's hand holding a delivery device according to an embodiment of the present invention.

FIG. 17A is an illustration of a person's hand holding a delivery device according to an embodiment of the present invention.

FIG. 17B is an illustration of a person's hand holding a delivery device according to an embodiment of the present invention.

FIG. 18 illustrates various alternative finger flange assemblies according to embodiments of the present invention.

FIG. 19 illustrates various alternative plungers according to embodiments of the present invention.

FIGS. 20A-20D are various schematic views of an alternative plunger according to an embodiment of the present invention.

### DETAILED DESCRIPTION OF THE INVENTION

The present invention will now be described in detail with reference to embodiments thereof as illustrated in the accompanying drawings. References to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

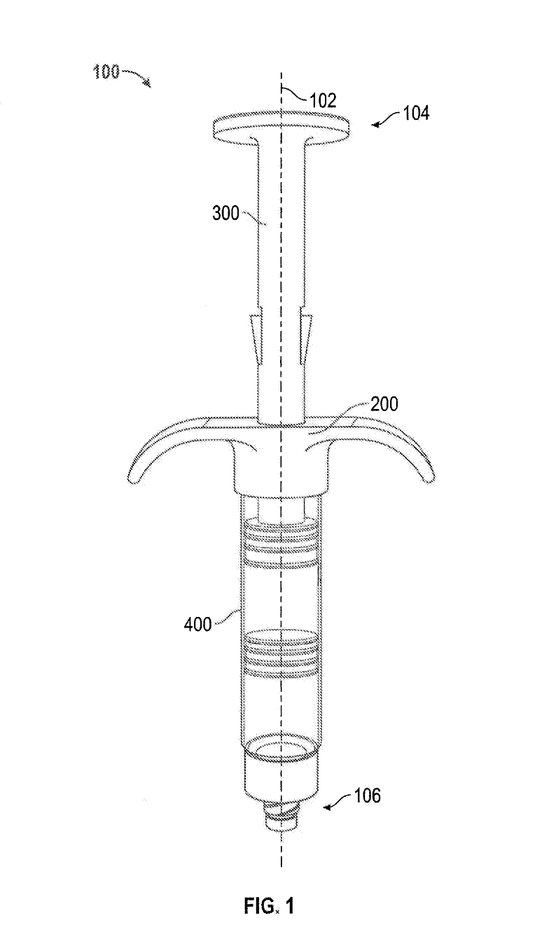

FIG. 1 depicts a delivery device 100 according to an embodiment of the present invention. Delivery devices, such as syringes, may generally be used as pumps to take in and/or expel liquids or gasses. Delivery devices may be used for a wide variety of medical and non-medical purposes. Medical uses for delivery devices, such as syringes, may include, for example, administering a therapeutic injection to a patient or withdrawing blood from a patient. Non-medical uses for delivery devices may include, for example, applying an adhesive compound while constructing an article of manufacture or injecting a flavored liquid into a food product while cooking. In one embodiment of the present invention, a syringe may be used to deliver blood clotting factor proteins via intravenous injections to an individual suffering from hemophilia. While the present discussion focuses primarily on the use of syringes for medical purposes, the delivery presently described herein may not be limited to syringes and may not be so limited in application.

As illustrated in the front view of FIG. 1, the syringe 100 generally includes a barrel 400, a finger flange assembly 200, and a plunger 300. The barrel 400 may be located at the distal end 106 of the syringe 100. The finger flange assembly 200 may be coupled to the barrel 400. The plunger 300 may be located at the proximal end 104 of the syringe 100, and the plunger 300 may pass through the finger flange assembly 200 to couple to the barrel 400. Features of the barrel 400, the finger flange assembly 200, and the plunger 300 are discussed in further detail below.

The proximal end 104 of the syringe 100 depicted in FIG. 1 is the upper-most end of the syringe 100, while the distal end 106 of the syringe 100 depicted in FIG. 1 is the lower-most end of the syringe 100. Accordingly, as used herein, the “proximal” direction or “proximal” side of an element can generally be considered to be the direction or side that is oriented toward the upper-most end of the plunger 300 of the syringe 100, while the “distal” direction or “distal” side of an element can generally be considered to be the direction or side that is oriented toward the lower-most end of the barrel 400 of the syringe 100, unless otherwise stated.

As further illustrated by FIG. 1, the syringe 100, or portions thereof, may define a central axis 102 (shown as a dashed line in FIG. 1) running down the center of the syringe 100 in the proximal-distal direction from the proximal end 104 to the distal end 106 of the syringe 100. In some embodiments, the syringe 100 may be symmetrical about one or more planes (i.e. reflection symmetry) or about one or more axes (i.e. rotation symmetry), such as the central axis 102. Compared to conventional syringes, which generally have asymmetrical finger flange assemblies and/or plungers, embodiments of the present invention that are symmetrical may allow users to quickly manipulate the syringe 100 without first having to realign the finger flange assembly 200 and/or the plunger 300 to an optimal alignment.

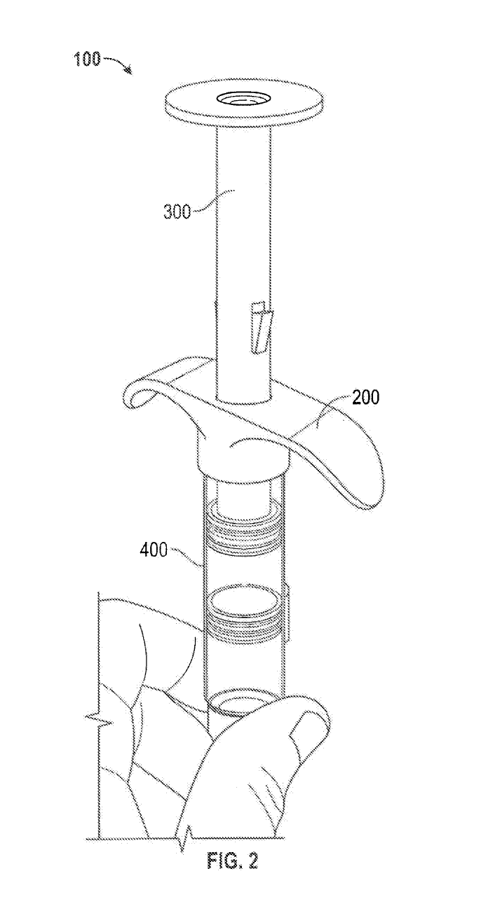

FIG. 2 illustrates the syringe 100 according to an embodiment of the present invention from a front perspective view from above. From this view, additional perspective on the relative dimensions and features of this exemplary syringe 100, including its barrel 400, finger flange assembly 200, and plunger 300 components, can be obtained.

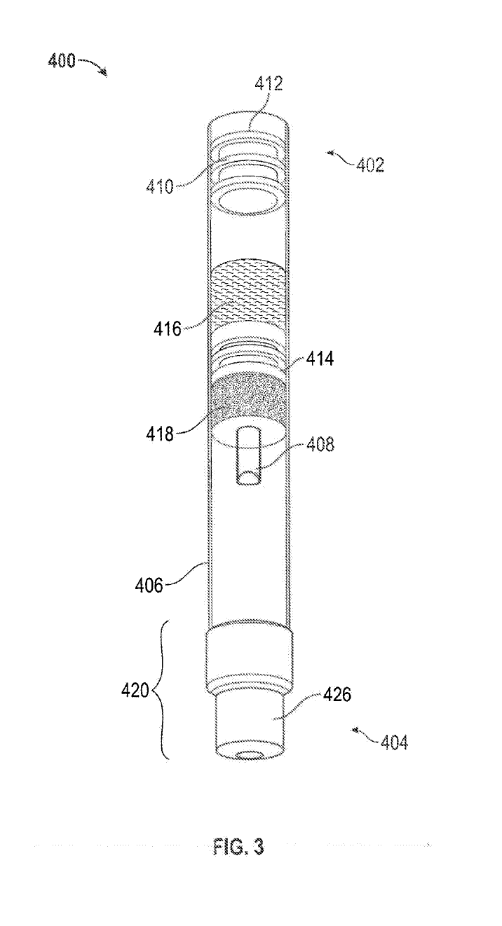

Turning first to the barrel 400 component of the syringe 100, FIG. 3 depicts a barrel 400 according to an exemplary embodiment of the present invention. The barrel 400 may be configured to hold one or more materials, such as liquids, gases, and/or solids. In the medical context, the barrel 400 may hold a medicament, such as a pharmaceutical composition, for later dispensation, or may be capable of holding blood drawn from a patient. For example, an individual afflicted with hemophilia may receive intravenous injections from the syringe 100 with a barrel 400 that holds certain blood clotting factor proteins necessary for treating hemophilia. In one embodiment, the barrel 400 may include a main body 406, one or more pistons (e.g. a proximal piston 410 and a distal piston 414), and a hub 420. In one embodiment, the barrel may be arranged in a manner similar to that disclosed in U.S. Pat. No. 4,874,381 to Vetter, the entirety of which is incorporated herein by reference thereto. In another embodiment, the barrel 400 may be, for example, a barrel such as those provided by Vetter Pharma-Fertigung GmbH & Co. KG of Ravensburg, Germany, and sold under the LYO-JECT brand.

The main body 406 of the barrel 400 of the syringe 100 may be primarily responsible for holding one or more materials (e.g. liquids, gases, and/or solids) inside the barrel 400. In one embodiment, the main body 406 may include multiple chambers for holding one or more materials in isolation from one another. The main body 406 may be one of many shapes or sizes, depending on the particular application. For example, the main body 406 may take the shape of a cylinder, a rectangular prism, a sphere, a cube, a cone, a pyramid, or combinations thereof. The main body 406 illustrated in FIG. 3 is substantially cylindrically shaped, and includes a single, central chamber. The main body 406 may have a total volume of, for example, between 0.1 and 50 ml. In some embodiments, the main body 406 may have a total volume of, for example, 0.1 ml, 0.5 ml, 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 20 ml, 25 ml, 30 ml, 35 ml, 40 ml, 45 ml, or 50 ml. The main body 406 of the syringe 100 may include visibly graduated marks and/or labels to indicate to a user of the syringe 100 the volume of material in the syringe 100 at a given time.

The main body 406 may be made from a variety of materials. In one embodiment, such as that depicted in FIG. 3, the main body 406 may be made from glass. In another embodiment, the main body may be made from one or more plastics such as, for example, polyvinylchloride, polytetrafluoroethylene, polyethersulfone, polyethylene, polyurethane, polyetherimide, polycarbonate, polyetheretherketone, polysulfone, polypropulene, cyclic olefin polymer, cyclic olefin copolymer, or combinations thereof. The main body 406, or portions thereof, may be transparent. In other embodiments, the main body 406, or portions thereof, may be translucent or opaque. In one embodiment, the main body 406 may be made by an injection molding manufacturing process wherein heated material is forced into a mold cavity where it cools and hardens to the configuration of the cavity.

In addition to the main body 406, the barrel 400 of the syringe 100 may also include one or more pistons (e.g. 410 or 414). The piston may be contained within and capable of moving relative to the main body 406. The motion of the piston within the main body 406 in a distal direction may cause materials held by the main body 406 to be expelled by the syringe 100. Conversely, the motion of a piston within the main body 406 in a proximal direction may cause materials held outside of the syringe 100 to be drawn into the main body 406. Pistons may operate by creating pressure differentials or vacuums that cause material to flow from an area of relatively high pressure to an area of relatively low pressure.

In one embodiment of the present invention, the main body 406 of the barrel 400 may include a single piston. In other embodiments, the main body 406 may include multiple pistons. FIG. 3 illustrates an embodiment where the main body 406 of the barrel 400 of the syringe 100 includes two pistons, a proximal piston 410 and a distal piston 414.

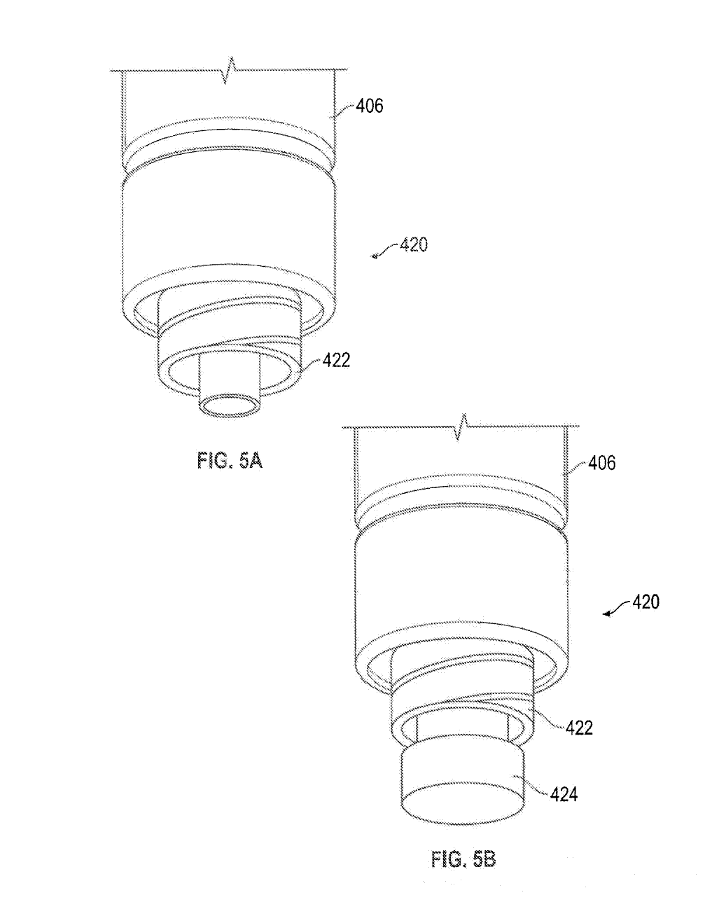

Pistons may be one of many shapes or sizes, depending on the particular application. The size and shape of a piston may depend on the corresponding size and shape of the main body 406 that the piston is contained within. For example, as shown in FIG. 3, if the main body 406 is generally cylindrically shaped, pistons, such as the proximal piston 410 and the distal piston 414, may also be generally cylindrically shaped. In other embodiments, conical or spherically shaped pistons may also be utilized with a generally cylindrically shaped main body 406. Some pistons, such as the proximal piston 410 and the distal piston 414 shown in FIG. 5, may include wings or rings for engaging the inner walls of the main body 406.

Pistons, such as the proximal piston 410 and the distal piston 414, may be made from a variety of materials. In one embodiment, pistons may be made from one or more plastics, such as those described above. In another embodiment, such as that depicted in FIG. 3, pistons may be made from one or more varieties of rubber. Suitable types of rubber may include, for example, natural rubber, polyurethane, silicone rubber, latex rubber, chlorobutyl rubber, or bromobutyl rubber.

In addition to the main body 406 and one or more pistons, the barrel 400 may further include a hub 420, as illustrated in FIG. 3. The hub 420 may be located at the distal end 404 of the barrel 400 of the syringe 100, and may include the orifice through which materials are capable of flowing into and out of the syringe 100. The hub 420 may be one of many shapes or sizes, depending on the particular application. In addition, the hub 420 may be made from a variety of materials, such as, for example, one or more of the plastics described above.

The hub 420 may consist of several subcomponents. In one embodiment, as illustrated in FIG. 3 the orifice (not shown) of the hub 420 may be temporarily covered by a closure 426 subcomponent. The closure 426 may be, for example, a screw cap, vent cap, or other temporary cap that can be removed and/or broken away from the hub 420. The closure 426 may be fixed to the main body of the hub 420 in such a way that accidental and premature removal of the closure 426 is unlikely.

The above-described components of the main body 406 of the barrel 400 of the syringe 100 may work together to retrieve, hold, and/or dispense materials to and from the syringe 100. For example, the main body 406 of the barrel 400 may hold a medicament, such as a pharmaceutical composition, for later dispensation out of the hub 420 of the syringe 100. As previously indicated, one or more pistons of the barrel 400 may enable movement of a medicament or other material through the syringe 100.

The barrel 400 of the syringe 100 illustrated in FIG. 3 includes a main body 406, a proximal piston 410 and a distal piston 414, and a hub 426. Such a main body 406 is capable of holding two separate materials in isolation from one another. In certain embodiments, the two separate materials may be a wide variety of liquids, gasses, and/or solids. In the embodiment depicted in FIG. 3, a liquid diluent 416 is held in the main body 406 between the proximal piston 410 and the distal piston 414, while a solid medicament 418 is held distally from the distal piston 414, between the distal piston 414 and the hub 420.

Certain medicaments, such as pharmaceutical compositions, may retain a longer shelf life when stored in certain states. For example, certain therapeutic proteins may be stored and kept for relatively long periods of time in a solid lyophilized (i.e. freeze-dried) state. However, prior to therapeutic use, such medicaments may need to be reconstituted and reactivated by mixing them with a diluent liquid.

The embodiments described herein are capable of being used with a wide variety of medicaments, including pharmaceutical compositions stored in a lyophilized state. The embodiments described herein are capable of being used with a wide variety of diluents, as necessary. In one embodiment, the syringe 100 barrel 400 may store a therapeutic in a liquid formulation for treating any disease or disorder that may be treated via an injection such as, for example, an intradermal, subcutaneous, intramuscular, or intravenous injection. In another embodiment, the syringe 100 barrel 400 may store one or more lyophilized proteins used in the treatment of bleeding disorders or conditions. Examples of the bleeding conditions or disorders include, but are not limited to, a bleeding coagulation disorder, hemarthrosis, muscle bleeding, oral bleeding, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, or bleeding in the illiopsoas sheath. In some aspects, the proteins in the syringe 100 can be used prophylactically or on-demand. In other aspects, the proteins in the syringe 100 can be used before, during, or after surgery.

In one embodiment, the medicament may be any therapeutic that can be administered in a liquid formulation. In some embodiments, medicaments may include small molecule biologics, large molecule biologics, and proteins. In one embodiment, the medicament may be a protein capable of treating a blood clotting disorder, such as a clotting factor. The protein may be lyophilized. The medicament may be stored in the syringe 100.

In one embodiment, one or more proteins that can be stored in the syringe 100 and may include, but are not limited to, FVIII coagulation factor proteins or FIX coagulation factor proteins. Additional clotting factor proteins that may be stored in the present syringe 100 include, for example, factor I (fibrinogen), factor II (prothrombin), Tissue factor, factor V (proaccelerin, labile factor), factor VII (stable factor, proconvertin), factor X (Stuart-Prower factor), factor XI (plasma thromboplastin antecedent), factor XII (Hageman factor), factor XIII (fibrin-stabilizing factor), VWF, prekallikrein (Fletcher factor), high-molecular-weight kininogen (HMWK) (Fitzgerald factor), fibronectin, antithrombin III, heparin cofactor II, protein C, protein S, protein Z, plasminogen, alpha 2-antiplasmin, tissue plasminogen activator (tPA), urokinase, plasminogen activator inhibitor-1 (PAI1), and plasminogen activator inhibitor-2 (PAI2). The barrel 400 may also store a liquid diluent 416 for reconstituting the lyophilized proteins such as, for example, sterile water for injection, a sodium chloride solution, saline, or a WFI solution.

Returning to FIG. 3, in this exemplary embodiment, a liquid diluent 416 is held in the main body 406 between the proximal piston 410 and the distal piston 414, while a solid medicament 418 is held distally from the distal piston 414, between the distal piston 414 and the hub 420. Because the proximal piston 410 and the distal piston 414 provide waterproof seals with the internal walls of the main body 406, the diluent 416 is held between the proximal piston 410 and the distal piston 414 in isolation from the solid medicament 418.

It should be noted that in other embodiments, other suitable dual-chamber syringe structures may be used wherein a liquid diluent and a medicament are held apart from one another by other means other than separation by a plunger within the barrel, including two parallel barrels.

As illustrated in FIGS. 1 and 2, and as explained in further detail below, a portion of the plunger 300 of the syringe 100 may be coupled to the barrel 400 by way of being attached to the proximal piston 410. With reference to FIG. 3, when so assembled, the plunger 300 may be capable of being driven distally downward to cause relative movement of the proximal piston 410 with respect to the main body 406 of the barrel 400. Distal movement of the proximal piston 410 may cause distal movement of the diluent 416, which in turn may cause distal movement of the distal piston 414.

Were the main body 406 of the barrel 400 of the syringe 100 to maintain a uniform circular cross sectional area along its cylindrical length, the liquid diluent 416 and solid medicament 418 may be held in isolation indefinitely and may not be capable of mixing. However, in one embodiment, as illustrated in FIG. 3, the main body 406 may include a bypass 408 that protrudes from a wall of the main body 406. The bypass 408 may be, for example, a bubble, tube, extension, or other pathway that extends beyond the regular profile of the main body 406 to allow materials held within the main body 406 to flow therethrough. The bypass 408 of FIG. 3 is a small half-cylinder extension that protrudes from the full cylindrical main body 406 of the barrel 400. In one embodiment, the bypass 408 may be arranged in a manner similar to that disclosed in U.S. Pat. No. 4,874,381 to Vetter, the entirety of which is incorporated herein by reference thereto. In another embodiment, the bypass 408 may be, for example, a bypass present in a barrel such as barrels provided by Vetter Pharma-Fertigung GmbH & Co. KG of Ravensburg, Germany, and sold under the LYO-JECT brand.

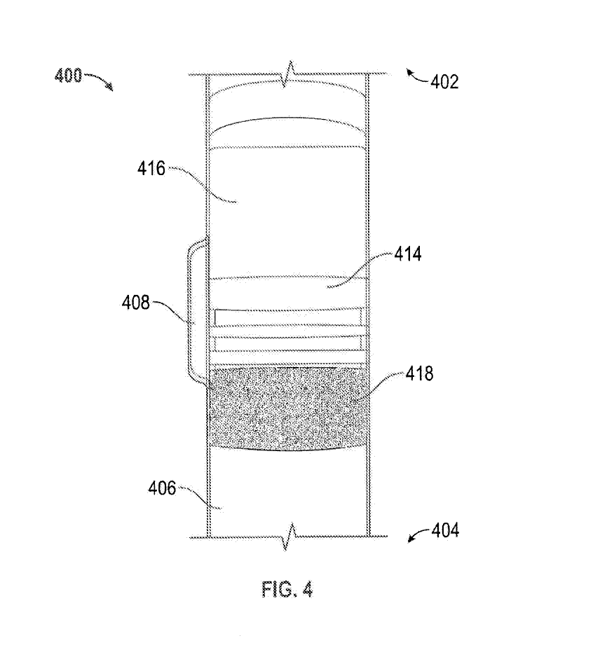

In FIG. 3, the distal piston 414 is at a position with respect to the bypass 408 such that the diluent 416 cannot flow past the distal piston 414 towards the solid medicament 418. However, as illustrated in FIG. 4, which is a close-up view of a portion of the barrel 400 of the syringe 100, as the plunger 300 is driven distally downward to cause distal movement of the proximal piston 410 and the distal piston 414, the distal piston 414 may reach a location in the main body 406 adjacent to the bypass 408 such that the diluent 416 is able to flow around the distal piston 414 to mix with and reconstitute the medicament 418, thus dissolving the medicament 418 into the liquid diluent 416. This is possible because the length of the distal piston 414 in the proximal-distal direction may be less than the length of the bypass 408 in the proximal distal direction. In this scenario the distal piston 414 will reach a location with respect to the bypass 498 where material may flow around both ends of the distal piston 414 via the bypass 408.

In one embodiment of the present invention, the main body 406 of the barrel 400 of the syringe 100 may be optimized and proportioned to allow for drawback during an injection. Draw-back is the act of pulling upward on the plunger 300 of a syringe 100 in the context of otherwise administering an injection. Drawback in useful in intravenous injections, such as, for example, injections to hemophilia patients, so that the user of the syringe 100 may confirm that the injection is in fact properly entering into a vein. Proper injection is confirmed when blood from the vein is drawn back into the syringe 100 temporarily. Were the injection not properly entering the vein, the drawback of blood would not be effective.

In the case of a dual chamber syringe 100 with a proximal piston 410, a distal piston 414, and a bypass 408, such as in the embodiment described above, draw-back may be desirable in the course of an injection. However, if the main body 406 of the barrel 400 of the syringe 100 is not optimally configured and proportioned, draw-back may be impeded by the fluid dynamics of the syringe 100. Specifically, once the distal piston 414 has moved distally past the bypass 408 in the injection cycle, the fluid dynamics of the main body 406 may be such that it is not possible to create a sufficient vacuum by moving the plunger 300 to draw the distal piston 414 back proximally beyond the bypass 408 where it started. If there is not enough space for the distal piston 414 to move proximally backward to allow for sufficient draw-back volume, this hindered ability to draw-back may be undesirable to certain users, such as those administering injections to a hemophilia patient.

In one embodiment of the present invention, the volume and/or length of the main body 406 of the barrel 400 of the syringe 100 is proportioned to be sufficiently voluminous and/or long such that sufficient draw-back is possible. More specifically, in an embodiment, the volume and/or length of the main body 406 of the barrel 400 of the syringe 100 distal to the bypass 408 is sufficiently large and/or lengthened such that sufficient draw-back is possible. For example, in various embodiments, the main body 406 may have a total volume of approximately 5 ml total and approximately 2.5 ml distal to the bypass 408, a total volume of approximately 2.5 ml total and approximately 1.25 ml distal to the bypass 408, or a total volume of approximately 1 ml total and approximately 0.5 ml distal to the bypass 408. In other embodiments, other total volumes and volumes distal to the bypass 408 may be appropriate, depending on the size of the main body used. In another embodiment, the main body 406 may have a length of between 80 mm and 120 mm total or between 42 mm and 64 mm distal to the bypass 408, between 82.5 mm and 87.5 mm total or between 44 mm and 46 mm distal to the bypass 408, or approximately 85 mm total or about 45 mm distal to the bypass 408.

Compared to conventional syringes 100, which may have main bodies 406 with total volumes and/or lengths or volumes and/or lengths distal to a bypass 408 that are different than those specified above, embodiments of the present invention with proportions as specified above may allow a user of the syringe 100 to readily confirm that an injection is in fact properly entering into a vein without struggling to draw back. In this way, a syringe 100 with a bypass 408 proportioned as specified above may provide a hemophilia patient self-administering an injection with an ergonomic structure that is easy to use and that does not require repeated realignments or manipulation struggles, which can lead to the generation of microbleeds.

Regardless of the volume or length of the main body 406, the number of pistons employed, or whether or not a medicament 418 needs to be reconstituted prior to evacuation from the syringe 100, materials may leave and enter the syringe through the hub 420 of the barrel 400 of the syringe 100.

FIGS. 5A and 5B are close-up perspective views of exemplary hubs 420 according to embodiments of the present invention. The hub 420 may be located at the distal end of the barrel 400 of the syringe 100, and may include the orifice through which materials are capable of flowing into and out of the syringe 100. As explained above, the hub 420 may consist of several subcomponents, and may be one of many shapes or sizes, depending on the particular application. In addition, the hub 420 may be made from a variety of materials, such as, for example, one or more of the plastics described above.

In one embodiment, as illustrated in FIG. 5A the hub 420 may distally terminate at a hub connector 422. The hub connector 422 may include the orifice for transferring materials in and out of the syringe, and may further include a fastener mechanism for securing the syringe 100 to an additional delivery component such as a needle, tube, or infusion set. As shown in FIG. 5A, the fastener mechanism of the hub connector 422 may be, for example, a plastic Luer lock or other suitable threaded, friction fit, or other fastening means.

As shown in FIG. 5B, a removable plug 424 may temporarily cover a portion of the hub connector 422. The removable plug 424 may be used to open and close the orifice of the hub connector 422. In one embodiment, the removable plug 424 may be a rubber plug that may temporarily be held on the hub connector 422 by friction fitting. In other embodiments, as illustrated in FIG. 3, both the hub connector 422 and the removable plug 424 may be covered by a closure 426. The closure 426 may be, for example, a screw cap or other temporary cap that may be removed and/or broken away from the hub 420. The closure 426 may be fixed to the main body of the hub 420 in such a way that accidental or premature removal of the closure 426 is unlikely. In this way, the closure 426 may act as the primary means to block the orifice of the hub 420 during storage and transportation, while the plug 424 may be a temporary block for the orifice once the closure 426 has been removed.

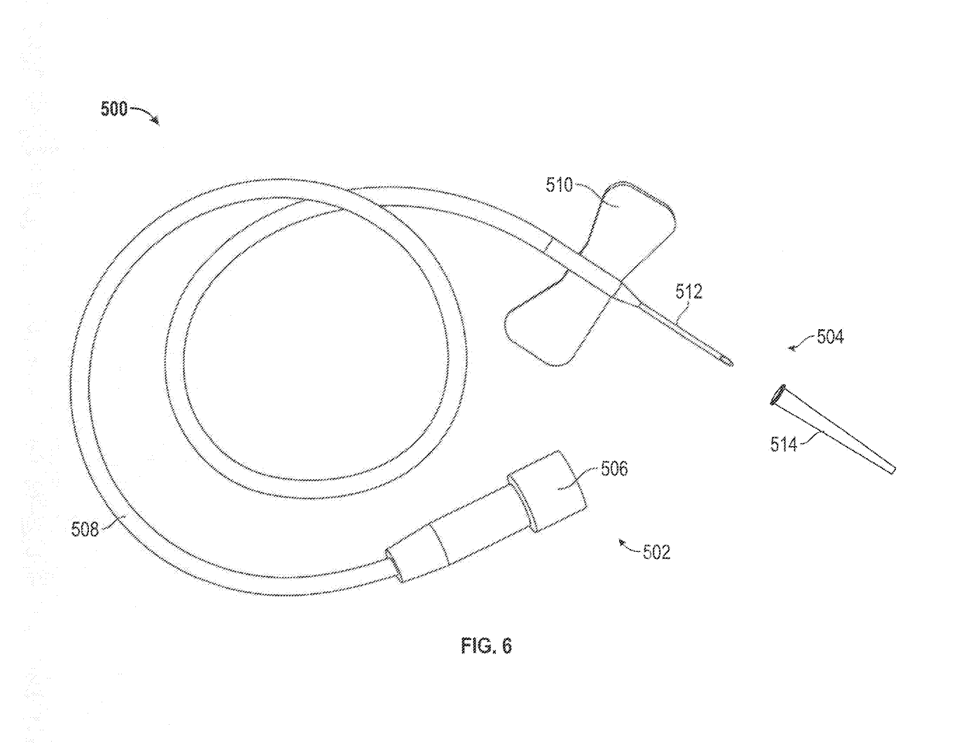

FIG. 6 illustrates a possible additional delivery component that may be attached to the hub 420 of the barrel 400 of the syringe 100, according to an embodiment of the present invention. As shown in FIG. 6, the exemplary additional delivery component may be an infusion set 500. The infusion set 500 may be one way to utilize the syringe 100 for transfer of material to or withdrawal of material from the human body without requiring the hub 420 of the syringe 100 (possibly fitted with a needle) to be brought into immediate proximity to the portion of the body to be treated. In this way, the use of the infusion set 500 may provide a patient or caregiver with improved ergonomics, versatility, and/or ease of use when working with the syringe 100.

As shown in FIG. 6, the infusion set 500 may include an infusion set connector 506 at its proximal end 502, a flexible tube 508 extending from the infusion set connector 506, a set of stabilizing wings 510 located just before the distal end 504 of the infusion set 500, a needle 512 at the distal end 504 of the infusion set 500, and a removable needle tip cover 514 for covering the needle 512.

The infusion set connector 506 may including a complementary fastener mechanism for mating with the fastener mechanism of the hub connector 422 of the barrel 400 of the syringe 100, such as, for example, a plastic Luer lock, Luer cone, Luer slip, or other suitable threaded, friction fit, or other fastening means.

The flexible tube 508 may be made from, for example, silicone or polyvinyl chloride tubing. In various embodiments, the flexible tube 508 may be between 5 mm and 50 mm long.

The set of stabilizing wings 510, located just before the distal end 504 of the infusion set 500, may be made of one or more of the above-mentioned plastics in one embodiment. The structure of the wings 510 may make it easier for a patient or caregiver to grasp and insert the needle 512 at a relatively shallow angle into a relatively small vein, such as a vein in the hand, wrist, or other locations where the vein is close to the surface of the skin. The wings 510 may also aid in keeping the needle 512 in place and to prevent damage to the patient's vessels if the patient moves during the syringe 100 procedure.

The needle 512 itself is used to pierce the patient's skin and to deliver materials to or withdraw materials from the patient's body. Suitable needles may include standard needles used for intradermal, subcutaneous, intramuscular, or intravenous injections, depending on the particular application. Finally, the needle 512 may be temporarily enclosed by a needle tip cover 514 for protecting the needle 512 and for preventing accidental needle sticks.

Because individuals affected by hemophilia are highly susceptible to accidental bleeding in everyday situations, stress points on tools, such as the infusion set 500, and stresses resulting from unnatural movements of the body required to insert the needle 512 into a vein or to receive the needle in one's vein have the potential to cause microbleeds. The use of an the infusion set 500 with a set of stabilizing wings 510 may help to make the insertion of the needle 512 a more ergonomics process, and to minimize movement of the needle 512 after insertion.

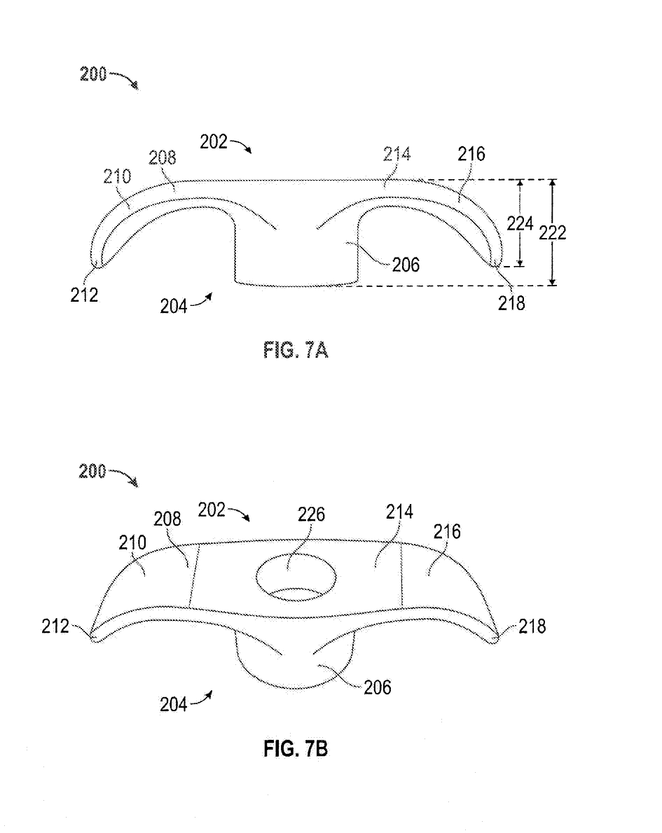

Turning now to the finger flange assembly 200 component of the syringe 100, FIGS. 7A, 7B, 8A, 8B, and 9 depict certain exemplary features of the finger flange assembly 200. The finger flange assembly 200 may generally be configured to receive portions of a patient or caregiver's fingers or hand to assist in gripping, steadying, or otherwise manipulating the syringe 100 when utilizing the syringe 100. As illustrated in FIGS. 1 and 2, the finger flange assembly 200 may be located distally from the barrel 400 of the syringe 100 and coupled to the barrel 400. In some embodiments, the plunger 300 of the syringe 100 may extend proximally from the finger flange assembly 200 and may pass through the finger flange assembly 200 to couple to the barrel 400.

The finger flange assembly 200 may be made from a variety of materials. In one embodiment, the finger flange assembly 200 may be made from one or more plastics such as, for example, polyvinylchloride, polytetrafluoroethylene, polyethersulfone, polyethylene, polyurethane, polyetherimide, polycarbonate, polyetheretherketone, polysulfone, polypropulene, cyclic olefin polymer, cyclic olefin copolymer, or combinations thereof. The finger flange assembly 200, or portions thereof, may be opaque. In other embodiments, the main body 406, or portions thereof, may be transparent or translucent. In one embodiment, the finger flange assembly 200 may be made by an injection molding manufacturing process.

In some embodiments, the finger flange assembly 200 may be a unitary structure manufactured as a unitary piece of material. In other embodiments, the finger flange assembly 200 may consist of two or more discrete pieces of material that are joined together to form the finger flange assembly 200 of the syringe 100. In one embodiment of the present invention, the finger flange assembly 200 may be integrally formed with the barrel 400 of the syringe 100 such that the finger flange assembly 200 and the barrel 400 are a unitary structure. Regardless of whether the finger flange assembly 200 is made of discrete components or whether it is integrally formed with the barrel 400, the finger flange assembly 200 or portions thereof may be releasably or permanently affixed to other components of the syringe 100 by fastener means such as, for example, an adhesive, threaded means, a friction fit, or other suitable means.

In the exemplary embodiment shown in FIGS. 7A, 7B, 8A, 8B, and 9, the finger flange assembly 200 may include a trunk 206 and one or more flanges, such as a first flange 208 and a second flange 214.

The trunk 206 of the finger flange assembly 200 may be centered about the central axis 102 of the syringe 100 and may be the portion of the finger flange assembly 200 that couples to the barrel 400 of the syringe. Such coupling can be seen, for example, in FIGS. 1 and 2. The trunk 206 may be one of many shapes or sizes, depending on the particular application. For example, the trunk 206 may take the general shape of a cylinder, a rectangular prism, a sphere, a cube, a cone, a pyramid, or combinations thereof. The trunk 206 illustrated in FIGS. 7A, 7B, 8B, and 9 is substantially cylindrically shaped. A substantially cylindrically shaped trunk 206 with relatively smooth edges may provide a hemophilia patient self-administering an injection with a satisfactory and safe structure to grasp that is free of stress points capable of leading to microbleeds.

In some embodiments of the present invention, one or more flanges may branch out from the trunk 206 of the finger flange assembly 200. In the embodiment depicted in FIGS. 7A, 7B, 8A, 8B, and 9, the first flange 208 and a second flange 214 each extend laterally from opposite sides of the finger flange assembly 200. In this way, the finger flange assembly 200 may be symmetrical about a plane that divides the finger flange assembly 200 evenly with the first flange 208 on one side of the plane and the second flange 214 on the other side of the plane. In other embodiments, lesser or greater than two finger flanges may be present, and the arrangement of finger flanges may or may not yield a symmetrical finger flange assembly 200.

Compared to conventional syringes, which may have asymmetrical finger flange assemblies, embodiments of the present invention with symmetrical finger flange assemblies 200 may allow users to quickly manipulate the syringe 100 without first having to realign the finger flange assembly 200 to an optimal alignment, or orient the syringe 100 in their hand so that their fingers “match” the configuration of the finger flange assembly 200. In this way, a symmetrical finger flange assembly 200 may provide a hemophilia patient self-administering an injection with an ergonomic structure that is easy to use and that does not require repeated realignments, which can lead to the generation of microbleeds.

The finger flange assembly 200 may be considered to have a proximal end 202 and a distal end 204. As indicated in FIGS. 7A and 7B, the proximal end 202 of the finger flange assembly 200 may be the end of the finger flange assembly 200 that includes a face facing the proximal end 104 of the syringe 100, which is the upward direction in the majority of the figures. As also indicated in FIGS. 7A and 7B, the distal end 204 of the finger flange assembly 200 may be the end or ends of the finger flange assembly 200 that include a faces or components facing the distal end 106 of the syringe 100, which is the downward direction in the majority of the figures.

FIG. 7A is a front view of the finger flange assembly 200 according to an embodiment of the present invention. In this embodiment, as the generally cylindrical trunk 206 extends proximally upward, a first flange 208 and a second flange 214 may each extend laterally from opposite sides of the finger flange assembly 200. The first flange 208 and a second flange 214 may gradually curve and taper outward from the trunk 206.

In an embodiment, as shown in FIG. 7A, the first flange 208 and the second flange 214 each curve toward the distal direction and terminate in a first flange tip 212 and a second flange tip 218. Along the way to the respective flange tips, the first flange 208 includes a first flange curve 210 region and the second flange 214 includes a second flange curve 216 region.

Compared to conventional syringe finger flange assemblies, which generally have finger flange assemblies that stick straight out with little or no curvature, embodiments of the present invention having a first flange 208 and a second flange 214 that gradually curve and taper outward from the trunk 206 may provide a hemophilia patient self-administering an injection with an ergonomic structure that is easy to use and grip during an injection. If a finger flange assembly includes no curvature, a user's fingers may tend to slip off of the finger flange assembly during the administration of an injection. However, if a finger flange assembly includes too severe of a curvature, a user's fingers may tend to undesirably get caught in the finger flange assembly during the administration of an injection, even at times when it may be desirable for the user to temporarily remove their fingers.

Repeated unnecessary realignments and re-grips or undesirable ensnaring of a user's fingers can lead to overall poor ergonomics and the generation of microbleeds. As described above, this repeated microbleed generation over time has the potential to cause severe damage to the body of a person affected by hemophilia. Given the need for regularly scheduled blood clotting factor protein injections for many hemophilia patients, an optimally designed finger flange assembly offering improved ergonomics, versatility, and/or ease of use is of great value.

The curvatures of the first flange curve 210 region and the second flange curve 216 region may vary according to the application. In one embodiment, the curvature of the first flange curve 210 region and the curvature of the second flange curve 216 region may be equal. In another embodiment, the curvature of the first flange curve 210 region and the curvature of the second flange curve 216 region may not be equal.

As indicated above, a symmetrical finger flange assembly 200 where the curvature of the first flange curve 210 region and the curvature of the second flange curve 216 region are equal may provide a hemophilia patient self-administering an injection with an ergonomic structure that is easy to use and that does not require repeated realignments, which can lead to the generation of microbleeds.

In one embodiment, the trunk 206 of the finger flange assembly 200 may define a central axis running down the center of the trunk 206 in the proximal-distal direction. This axis may be coextensive with the central axis 102 of the syringe 100 depicted in FIG. 1. If the first flange 208 and the second flange 214 are symmetrical about the central axis 102, the first flange tip 212 and the second flange tip 218 may each be equidistant from the central axis 102.

In another embodiment, as illustrated in FIG. 7A, the distance between the first flange tip 212 and the proximal-most extent of the finger flange assembly 200 (i.e. the flat top surface depicted in FIG. 7A), as measured parallel to the central axis 102, is equal to the distance between the second flange tip 218 and the proximal-most extent of the finger flange assembly 200, as measured parallel to the central axis 102. This distance, which may be referred to and illustrated as the flange tip height 224, may be equal to, for example, between 5 mm and 20 mm, between 10 mm and 15 mm, or approximately 12.5 mm. However, in some embodiments, the flange tip height 224 may be substantially shorter or substantially longer to suit the needs of individuals having smaller than average hands (e.g. small children) or larger than average hands, respectively.

If the flange tip height 224 is too short, in some embodiments of the present invention, a user's fingers may tend to slip off of the finger flange assembly during the administration of an injection. However, if the flange tip height 224 is too long, a user's fingers may tend to undesirably get caught in the finger flange assembly during the administration of an injection, even at times when it may be desirable for the user to temporarily remove their fingers. As explained above, repeated unnecessary realignments and re-grips or undesirable ensnaring of a user's fingers can lead to overall poor ergonomics and the generation of microbleeds in hemophilia patients.

As further illustrated in FIG. 7A, the distance between the distal-most extent of the trunk 206 and the proximal-most extent of the finger flange assembly 200 (i.e. the flat top surface depicted in FIG. 7A), as measured parallel to the central axis 102, may be referred to and illustrated as the assembly height 222. In various embodiments, the assembly height 222 may be equal to, for example, between 7.5 mm and 22.5 mm, between 12.5 mm and 17.5 mm, or approximately 15 mm. However, in some embodiments, the assembly height 222 may be substantially shorter or substantially longer to suit the needs of individuals having smaller than average hands (e.g. small children) or larger than average hands, respectively.

FIG. 7B is a front perspective view from above of the finger flange assembly 200 according to an embodiment of the present invention. In this embodiment, the arrangement and curvature of the first flange 208 and the second flange 214 of an exemplary embodiment of the finger flange assembly 200 are further illustrated. In addition, it can be seen that the finger flange assembly 200 may include an aperture running in the proximal-distal direction through the finger flange assembly 200. In an embodiment, the aperture may run in the proximal-distal direction down the central axis 102 of the syringe 100. As shown in FIG. 7B, the proximal end 202 of the finger flange assembly 200 may include a proximal aperture opening 226 at one end of the aperture.

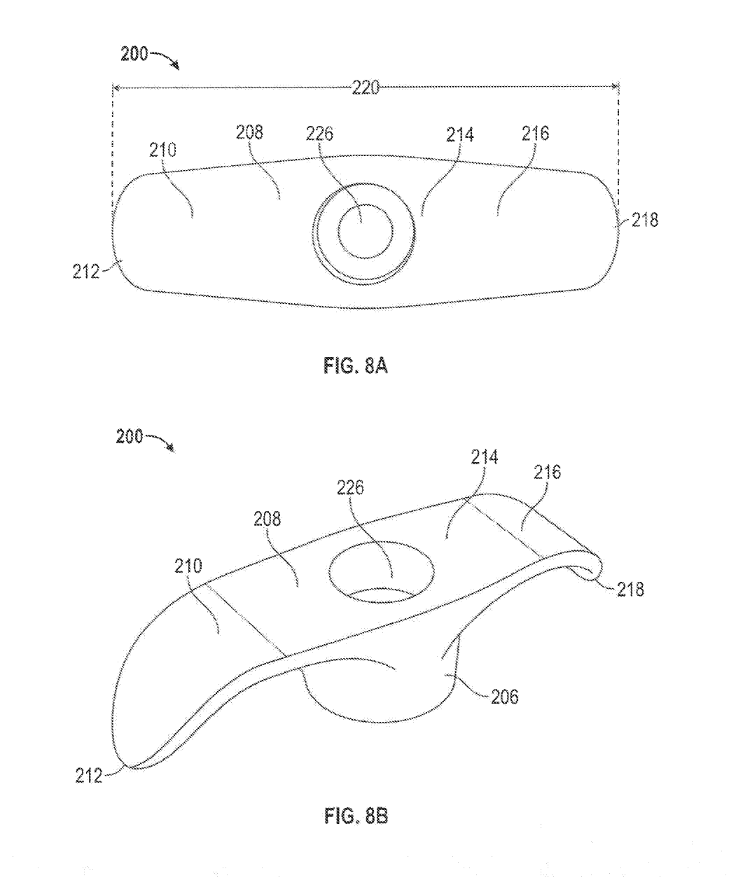

FIG. 8A is a top view of the finger flange assembly 200 according to an embodiment of the present invention. In this embodiment, the aperture can bee seen in additional detail. The proximal aperture opening 226 in FIG. 8A is circular. The distal end 204 of the finger flange assembly 200 may terminate in the trunk 206 which may include a distal aperture opening, which may be sized to mate with the proximal end 402 of the barrel 400 of the syringe 100. In one embodiment, the distal aperture opening may include a complementary fastener mechanism for mating with the barrel 400 of the syringe 100, such as, for example, a suitable threaded, friction fit, or other fastening means. In some embodiments, the proximal aperture inner diameter may be less than the distal aperture inner diameter. In alternate embodiments, the aperture may take non-cylindrical, non-circular shapes.

As further illustrated by FIG. 8A, in one embodiment, the first flange 208 and the second flange 214 may each taper and become narrower as they extend laterally toward their respective first flange tip 212 and second flange tip 218. In some embodiments the degree of the taper may be constant and gradual, while in other embodiments the degree of the taper may change. The first flange tip 212 and second flange tip 218 may terminate in lateral-most ends that are, for example, pointed, round, squared off, or a variety of other shapes and configurations.

If the degree of taper is too great, in some embodiments of the present invention, a user's fingers may tend to slip off of the finger flange assembly during the administration of an injection. However, if the degree of taper is too subtle, a user's fingers may tend to undesirably get caught in the finger flange assembly during the administration of an injection, even at times when it may be desirable for the user to temporarily remove their fingers. As explained above, repeated unnecessary realignments and re-grips or undesirable ensnaring of a user's fingers can lead to overall poor ergonomics and the generation of microbleeds in hemophilia patients.

In addition, if the first flange tip 212 and second flange tip 218 terminate in lateral-most ends that present overly sharp edges, these edges may act as stress points that have the potential to cause microbleeds in an individual affected with hemophilia as the individual interacts with the syringe 100. However, a relatively narrow first flange tip 212 and second flange tip 218 may allow the user to remove their fingers from the finger flange assembly 200 more easily as desired.

In one embodiment, as shown in FIG. 8A, the distance between the first flange tip 212 and the second flange tip 218, as measured perpendicular to the central axis, may be referred to and illustrated as the assembly width 220 of the finger flange assembly 200. The assembly width 220 may be, for example, between 57 mm and 72 mm, between 62 mm and 67 mm, or approximately 64.5 mm. However, in some embodiments, the assembly width 220 may be substantially shorter or substantially longer to suit the needs of individuals having smaller than average hands (e.g. small children) or larger than average hands, respectively.

If the assembly width 220 is too short, in some embodiments of the present invention, a user's may not be able to comfortably fit all of the desired finger or fingers into the finger flange assembly during the administration of an injection. However, if the assembly width 220 is too long, use of the finger flange assembly may become cumbersome and the flange tips may act as stress points.

With reference to the distances illustrated in both FIGS. 7A and 8A, in on embodiment of the present invention, the ratio of the flange tip height 224 to the assembly width 220 of the finger flange assembly 200 is between 1:2 and 1:8, between 1:4 and 1:6, or approximately 1:5.

In some embodiments, these ratios may allow a user to comfortably and effectively grasp the finger flange 200 of the syringe 100 in their hand without repeated unnecessary realignments and re-grips or undesirable ensnaring of a user's fingers, thus avoiding poor ergonomics and the generation of microbleeds in hemophilia patients.

In some embodiments, while the flange tip height 224 and assembly width 220 may be substantially shorter or substantially longer than those described above to suit the needs of individuals having smaller than average hands (e.g. small children) or larger than average hands, respectively, the overall ratio of between 1:2 and 1:8, between 1:4 and 1:6, or approximately 1:5 may be maintained. In other words, the overall configurations of some embodiments of the present invention may be proportionally scalable when compared to some of the embodiments described above as determined by user needs.

FIG. 8B is an isometric view of a finger flange assembly 200 according to an embodiment of the present invention. In this embodiment, the arrangement and curvature of the first flange 208 and the second flange 214 of an exemplary embodiment of the finger flange assembly 200 are further illustrated.

FIG. 9 is a side view of a finger flange assembly according to an embodiment of the present invention. In this illustration, the first finger flange 208 of the finger flange assembly 200 is projecting outward toward the viewer viewing the finger flange assembly 200 from the side. The assembly height 222 and the flange tip height 224 are illustrated in FIG. 9 in addition to FIG. 7A. The first flange tip 212 of the first flange 208 can also be seen curving downward in the distal direction toward the distal end 204 of the finger flange assembly 200 and toward the barrel 400 of the syringe.

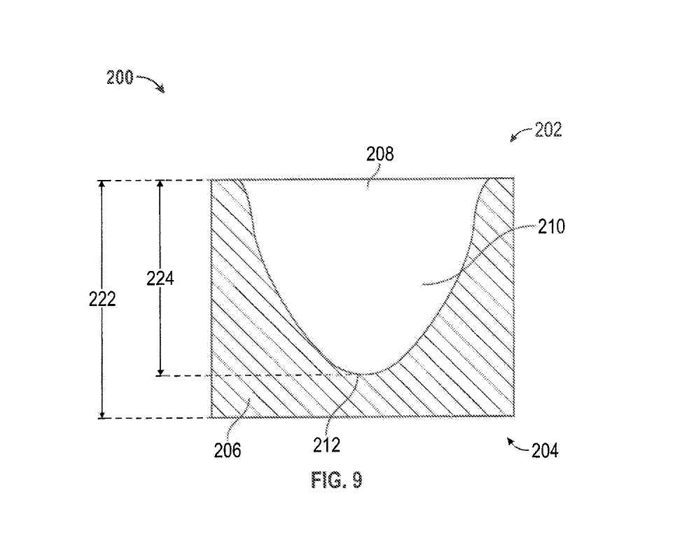

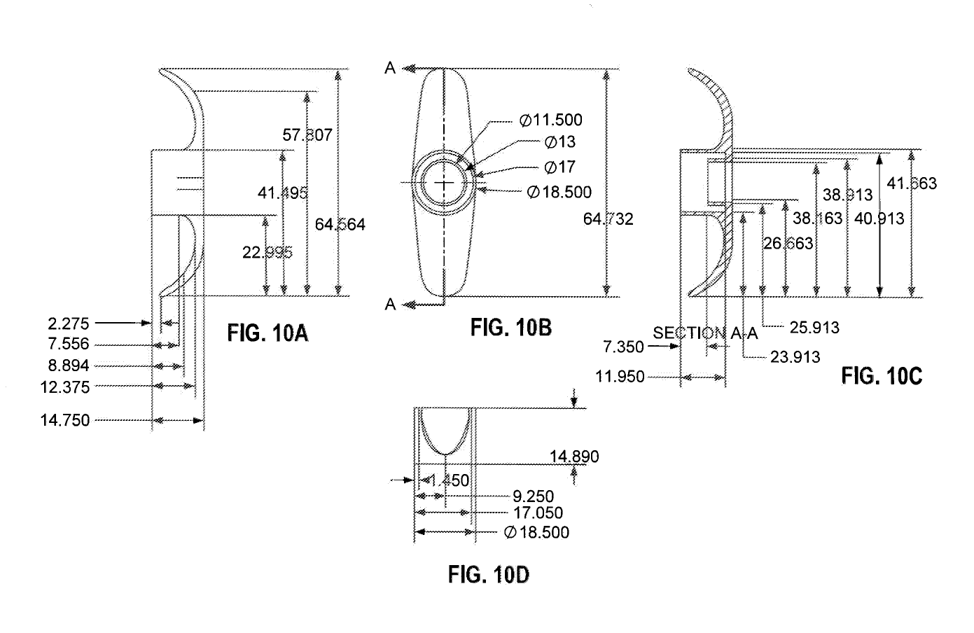

FIGS. 10A-10D are various schematic views of a finger flange assembly 200 according to an embodiment of the present invention. Exemplary measurements of portions of the finger flange assembly 200 are provided in millimeters.

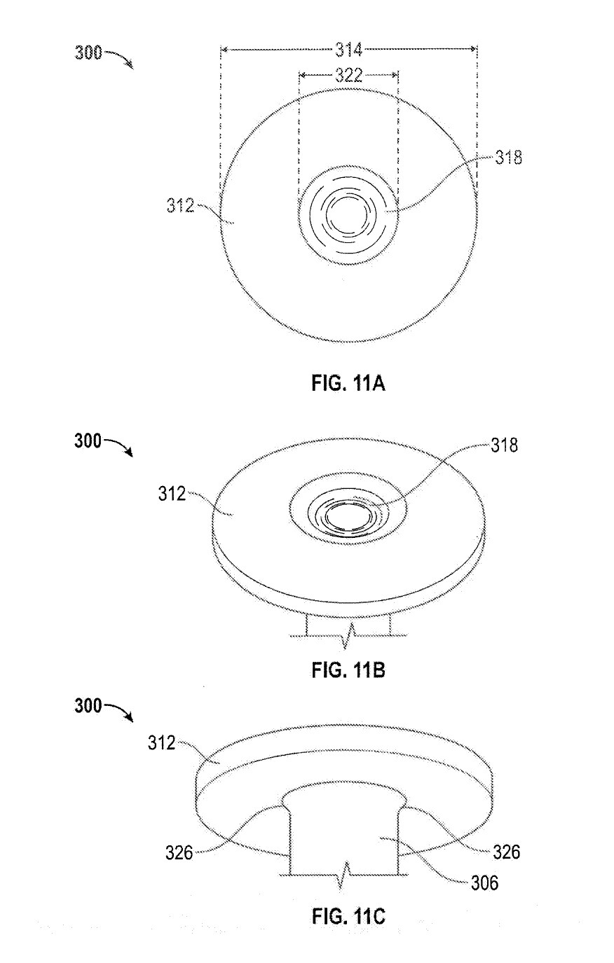

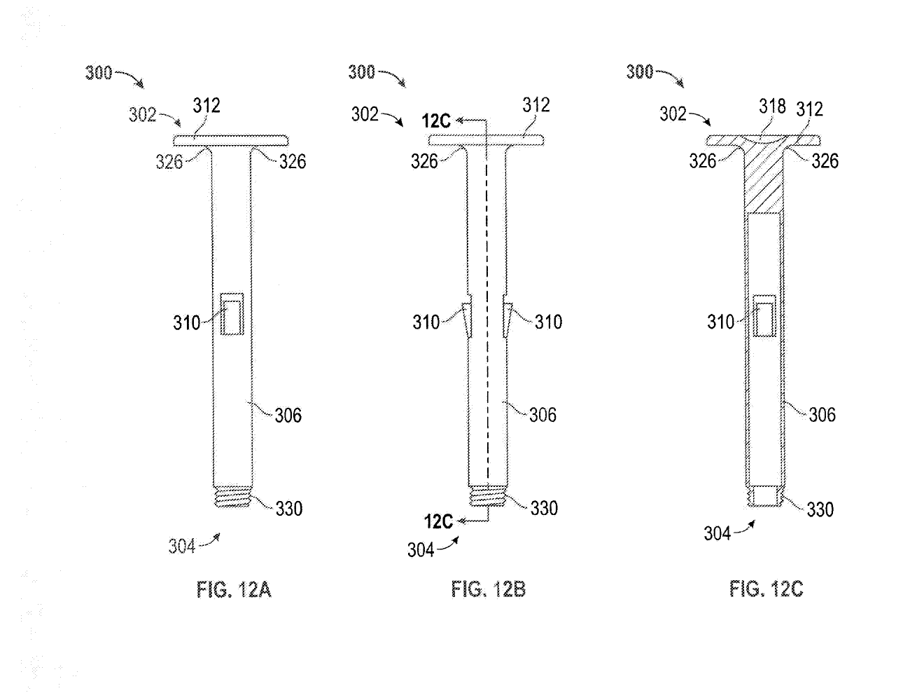

Turning now to the plunger 300 component of the syringe 100, FIGS. 11A-11C, 12A-12C, and 13 depict certain exemplary features of the plunger 300. In some embodiments, the plunger 300 may be capable of being driven distally downward by a patient or caregiver's thumb, fingers, or hand to cause the material held by the barrel 400 to be expelled. In other embodiments, the plunger 300 may be pulled proximally upward by a patient or caregiver's thumb, fingers, or hand to draw material into the barrel 400. In one embodiment, as illustrated, for example, in FIGS. 12A-12C, the plunger 300 may include a head 312 and a rod 306. As illustrated in FIGS. 1 and 2, in some embodiments, the plunger 300 of the syringe 100 may extend proximally from the finger flange assembly 200 in one direction, and may pass through the finger flange assembly 200 in the other direction to couple to the barrel 400.

The plunger 300 may be made from a variety of materials. In one embodiment, the plunger 300 may be made from one or more plastics such as, for example, polyvinylchloride, polytetrafluoroethylene, polyethersulfone, polyethylene, polyurethane, polyetherimide, polycarbonate, polyetheretherketone, polysulfone, polypropulene, cyclic olefin polymer, cyclic olefin copolymer, or combinations thereof. The plunger 300, or portions thereof, may be opaque. In other embodiments, the plunger 300, or portions thereof, may be transparent or translucent. In one embodiment, the plunger 300 may be an injection molding manufacturing process.

In some embodiments, the plunger 300 may be a unitary structure manufactured as a unitary piece of material. In other embodiments, the plunger 300 may consist of two or more discrete pieces of material that are joined together to form the plunger 300 of the syringe 100.

In the exemplary embodiment shown in FIGS. 11A-11C, 12A-12C, and 13, the plunger 300 may include a head 312 and a rod 306. The head 312 may serve as a surface upon which to apply force to drive the plunger 300 distally downward or to pull the plunger 300 proximally upward. The rod 306 may serve as the shaft by which the force applied to the head 312 is transmitted to the plunger of the barrel 400, or vice versa.

FIG. 11A is a top view of the plunger 300 according to an embodiment of the present invention. In this embodiment, the head 312 of the plunger 300 is circular when viewed from above. In other embodiments, the head 312 of the plunger 300 may be triangular, square, oval, or a variety of other shapes when viewed from above. In embodiments where the head 312 of the plunger 300 is circular when viewed from above, the circular head 312 may have a head diameter 314. The head diameter 314 may be, for example, greater than 25 mm. However, in some embodiments, the head diameter 314 may be substantially shorter or substantially longer to suit the needs of individuals having smaller than average hands (e.g. small children) or larger than average hands, respectively.

In embodiments where the head 312 of the plunger 300 is circular, the symmetry of the circular head may prevent a user from having to realign the head for optimal depression and withdrawal of the head during injection and drawback. In addition, if the head diameter 314 is too small, in some embodiments of the present invention, a user's finger or thumb may tend to slip off of the finger flange assembly during the administration of an injection. Repeated unnecessary realignments and re-grips of a plunger 300 can lead to overall poor ergonomics and the generation of microbleeds in hemophilia patients.

The top view of the plunger 300 in FIG. 11A also depicts a dimple 318 situated on the surface of the head 312 of the plunger 300. Heads 312 of plungers 300 according to embodiments of the present invention may include one or more dimples 318. Dimples 318 may be capable of providing increased friction, texture, structural differentiation, or other characteristics of the head 312 of the plunger 300 such that improved ergonomics, versatility, and/or ease of use are provided.

Dimples 318 may take on a variety of shapes and sizes. For example, dimples 318 may have circular, triangular, square, oval, spherical, cylindrical, conical, cubical, or other dimensions. Dimples 318 may also be placed on a variety of locations around the surface of the head 312. In the embodiment of FIG. 11A, a single, spherical dimple 318 is centered on the proximal surface 302 of the head 312. In some embodiments, the remainder of the proximal surface 302 of the head 312 beyond the dimple is entirely flat, while in other embodiments the remainder of the proximal surface 302 of the head 312 beyond the dimple may have curved, ridged, angled, or have another form of non-flat surface. An ergonomically structured head 312 and dimple 318 can greatly improve improved ergonomics, gripping, and ease of use of the plunger 300, which may ultimately lead to prevention of the generation of unnecessary microbleeds in hemophilia patients.

As further illustrated by FIG. 11A, the spherical dimple 318 may have a dimple diameter 322, where the dimple diameter 322 is defined as the diameter of the spherical dimple 318 as measured at the proximal surface 302 of the head 312. The dimple diameter 322 may be, for example, between 6 mm and 14 mm, between 8 mm and 12 mm, or approximately 10 mm. However, in some embodiments, the dimple diameter 322 may be substantially shorter or substantially longer to suit the needs of individuals having smaller than average hands (e.g. small children) or larger than average hands, respectively.

In one embodiment of the present invention, the ratio of the head diameter 314 to the dimple diameter 322 may be between 2:1 and 3:1 or between 1.0:1 and 1.5:1.

Depending on the size of the head 312 and the particular application, if the dimple diameter 322 is too small, in some embodiments of the present invention, the benefits of the dimple such as, for example, increased friction, texture, structural differentiation may be lost. Likewise, if the dimple diameter 322 is too large, these same benefits may also be lost. As explained above, repeated unnecessary realignments and re-grips of a plunger 300 can lead to overall poor ergonomics and the generation of microbleeds in hemophilia patients.

In some embodiments, while the head diameter 314 and dimple diameter 322 may be substantially shorter or substantially longer than those described above to suit the needs of individuals having smaller than average hands (e.g. small children) or larger than average hands, respectively, the overall ratio of between 2:1 and 3:1 or between 1.0:1 and 1.5:1 may be maintained. In other words, the overall configurations of some embodiments of the present invention may be proportionally scalable when compared to some of the embodiments described above as determined by user needs.

FIG. 11B is a front perspective view from above of the plunger 300 according to an embodiment of the present invention. In this embodiment, the arrangement and geometry of the head 312 and the spherical dimple 318 of an exemplary embodiment of the plunger 300 are further illustrated. In addition, it can be seen that in an embodiment, the head 312 of the plunger 300 may be a substantially circular disc-shaped head 312.

FIG. 11C is a front perspective view from below of the plunger 300 according to an embodiment of the present invention. Once again, the arrangement and geometry of the head 312 of an exemplary embodiment of the plunger 300 are further illustrated. The head 312 of the plunger 300 may be a substantially circular disc-shaped head 312. In addition, it can be seen that in an embodiment, the external boundary between the head 312 of the plunger 300 and the rod 306 of the plunger 300 may be defined by a circular incurvate edge 326. In some embodiments, the external boundary between the head 312 of the plunger 300 and the rod 306 of the plunger 300 may be defined by more linear edges that are not incurvate. In other embodiments, the external boundary between the head 312 of the plunger 300 and the rod 306 of the plunger 300 may be defined by edges that are not circular in nature, though this may or may not depend on the shape of the rod 306 of the plunger.

In some embodiments, the relative smoothness of the incurvate edge 326 as opposed to a more conventional linear edge may provide some level of protection against the generation of microbleeds in hemophilia patients, as well as increased friction, texture, structural differentiation on the bottom side of the head 312 of the syringe.

Additional features of the plunger 300 according to embodiments of the present invention are illustrated by the side, front and sectional views of an exemplary plunger 300 in FIGS. 12A-12-C. FIG. 12A is a side view of the plunger 300 according to an embodiment of the present invention. The plunger 300 includes a head 312 at the proximal end 302 of the plunger 300 and a rod 306 extending distally from the head 312 toward a distal end 304. The external boundary between the head 312 of the plunger 300 and the rod 306 of the plunger 300 may be defined by the circular incurvate edge 326.

In one embodiment, the plunger 300 may include a plunger connector 330 at its distal end 304 for engaging a proximal piston connector 412 of the proximal piston 410 (such as the proximal piston 410 illustrated in FIG. 3). In one embodiment, the plunger connector 330 may be a fastener including a helical ridge thread wrapped around a cylinder, and the proximal piston connector 412 may be a void in the proximal end of the proximal piston 410 with complementary threading for receiving and holding the plunger connector 330 after the plunger connector 330 is screwed into the proximal piston connector 412.

The rod 306 of the plunger 300 running from the head 312 to the plunger connector 330 may be a cylinder having a substantially uniform diameter throughout most of its length, as illustrated in FIG. 12A. In one embodiment, the rod 306 may be hollow, while in another embodiment the rod 306 may be filled. In an embodiment, the rod 306 of the plunger 300 may include one or more brakes 310 extending laterally outward from the main body of the rod 306. A single brake 310 is shown in FIG. 12A.

FIG. 12B is a front view of the plunger 300 according to an embodiment of the present invention. Many of the same features of the exemplary plunger 300 illustrated in FIG. 12A are also illustrated in FIG. 12B. However, in FIG. 12B, two oppositely opposed brakes 310 can be seen extending laterally from opposite sides of the rod 306 of the plunger 300. In one embodiment of the present invention, the brakes 310 may act to lock the plunger 300 into place in a depressed position after the plunger 300 has been distally depressed to a point where the brakes 310 have passed a complementary brake-stop mechanism in the finger flange assembly 200 of the syringe 100. In other embodiments where locking the plunger 300 into place in a depressed position after the plunger 300 has been distally depressed is not desired, the brakes 310 may be omitted from the rod 306.

FIG. 12C is a sectional view of the plunger 300 of FIG. 12B taken along the line A-A according to an embodiment of the present invention. The sectional view of FIG. 12C illustrates the spherical dimple 318 of the head 312 of the plunger 300 in cross section, which appears as an incomplete portion of a circle. The sectional view of FIG. 12C illustrates an embodiment where a portion of the rod 306 is hollow.

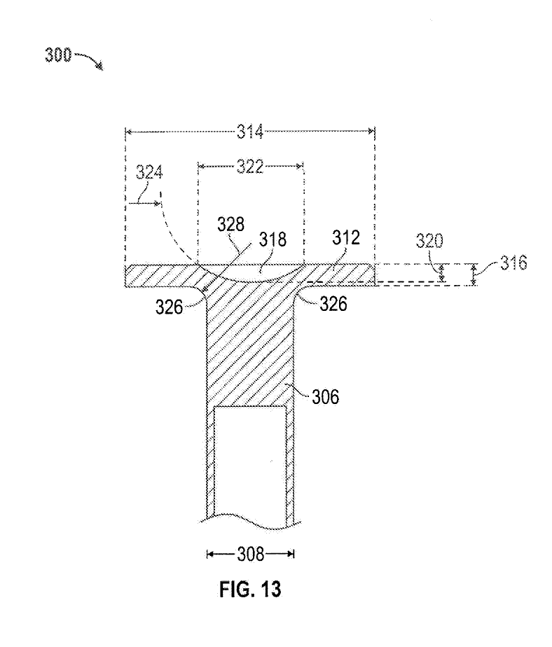

FIG. 13 is a close-up sectional view of a portion of the plunger of FIG. 12B taken along the line A-A according to an embodiment of the present invention. This view illustrates several additional features of embodiments of the plunger 300 according to the present invention.

As illustrated in FIG. 13, the diameter of the head 312 at its widest portion may be referred to and illustrated as the head diameter 314 of the head 312 of the plunger 300. In an embodiment, the head diameter 314 may be, greater than 25 mm. As further illustrated, the diameter of the rod 306 at a level just distal to the incurvate edge 326 may be referred to and illustrated as the rod diameter 308 of the plunger 300. The rod diameter 308 may be, for example, approximately 10 mm. In one embodiment of the present invention, the ratio of the head diameter 314 to the rod diameter 308 may be greater than 2.5:1. Depending on the size of the rod 306 and the particular application, if the head diameter 314 is too small, in some embodiments of the present invention, the ability of a user to firmly and easily grip the plunger with a thumb, finger, or palm of their hand may be lost. Likewise, if the head diameter 314 is too large these same benefits may also be lost. As explained above, repeated unnecessary realignments and re-grips of a plunger 300 can lead to overall poor ergonomics and the generation of microbleeds in hemophilia patients.

In some embodiments, while the head diameter 314 and rod diameter 308 may be substantially shorter or substantially longer than those described above to suit the needs of individuals having smaller than average hands (e.g. small children) or larger than average hands, respectively, the overall ratio of greater than 2.5:1 may be maintained. In other words, the overall configurations of some embodiments of the present invention may be proportionally scalable when compared to some of the embodiments described above as determined by user needs.

As further illustrated in FIG. 13, depth of the disc of the circular disc-shaped head 312 of the plunger 300 may be referred to and illustrated as the head depth 316 of the head 312 of the plunger 300. In an embodiment, the head depth 316 may be between 2 mm 3 mm. As is also illustrated, the radius of curvature of the spherical dimple 318 may be referred to and illustrated as the dimple radius of curvature 324. In an embodiment, the dimple radius of curvature 324 may be between 7.5 mm and 12.5 mm, or approximately 10 mm. Furthermore, the depth of the spherical dimple 318 extending downward into the head 312 of the plunger 300 may be referred to and illustrated as the dimple depth 320. In an embodiment, the dimple depth 320 may be between 0.1 mm and 2.9 mm, or approximately 2 mm.

Depending on the size of the head 312 and the particular application, if the dimple 318 is too small, in some embodiments of the present invention, the benefits of the dimple such as, for example, increased friction, texture, structural differentiation may be lost. Likewise, if the dimple 318 is too large, these same benefits may also be lost. As explained above, repeated unnecessary realignments and re-grips of a plunger 300 can lead to overall poor ergonomics.

In some embodiments, the dimple measurements may be substantially shorter or substantially longer to suit the needs of individuals having smaller than average hands (e.g. small children) or larger than average hands, respectively, and may be proportionally scalable when compared to some of the embodiments described above as determined by user needs.

In some embodiments, the spherical nature of the dimple 318 may add to the structural differentiation of this component, and may be capable of creating a suction or vacuum affect against the surface of a user's thumb, finger or the palm of their hand when depressed on the surface of the dimple, thus increasing the grip on the head 312 of the plunger 300.

As also shown in FIG. 13, the radius of curvature of the incurvate edge 326 may be referred to and illustrated as the incurvate edge radius of curvature 328 of the plunger 300. In an embodiment, the incurvate edge radius of curvature 328 may be between 1 mm and 3 mm, or approximately 2 mm, or between 6 mm and 10 mm, or approximately 8 mm.

In some embodiments, the relative smoothness of the incurvate edge 326 as opposed to a more conventional linear edge may provide some level of protection against the generation of microbleeds in hemophilia patients, as well as increased friction, texture, structural differentiation on the bottom side of the head 312 of the syringe, particularly within certain tolerances.

In some embodiments, the incurvate edge measurements may be substantially shorter or substantially longer to suit the needs of individuals having smaller than average hands (e.g. small children) or larger than average hands, respectively, and may be proportionally scalable when compared to some of the embodiments described above as determined by user needs.

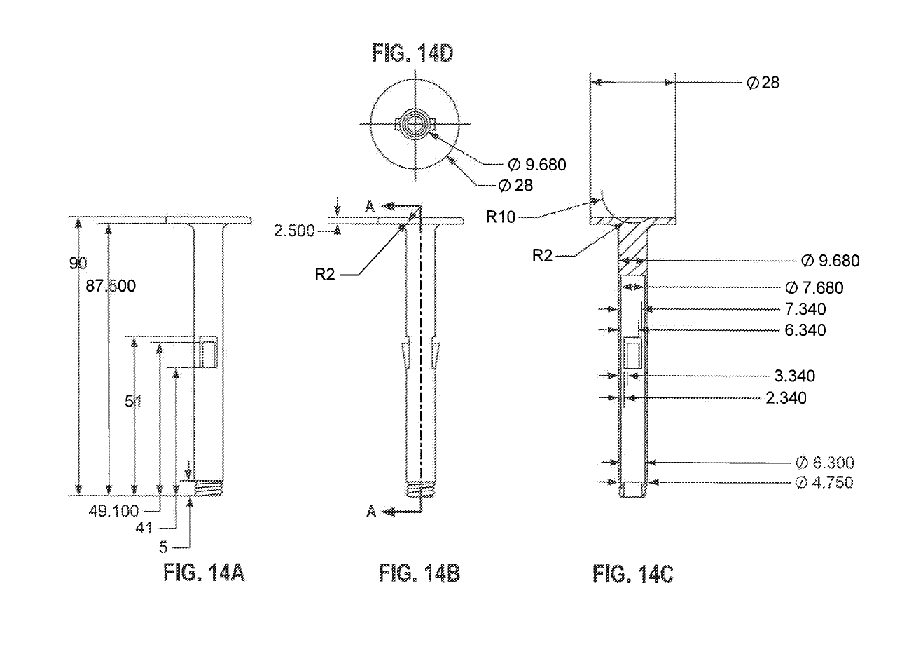

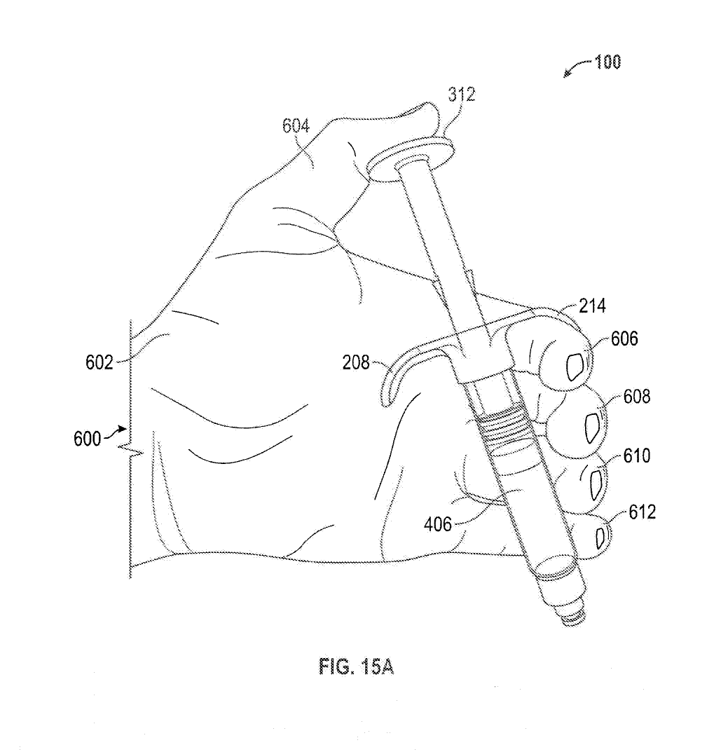

FIGS. 14A-14D are various schematic views of the plunger 300 according to an embodiment of the present invention. Exemplary measurements of portions of the plunger 300 are provided in millimeters.

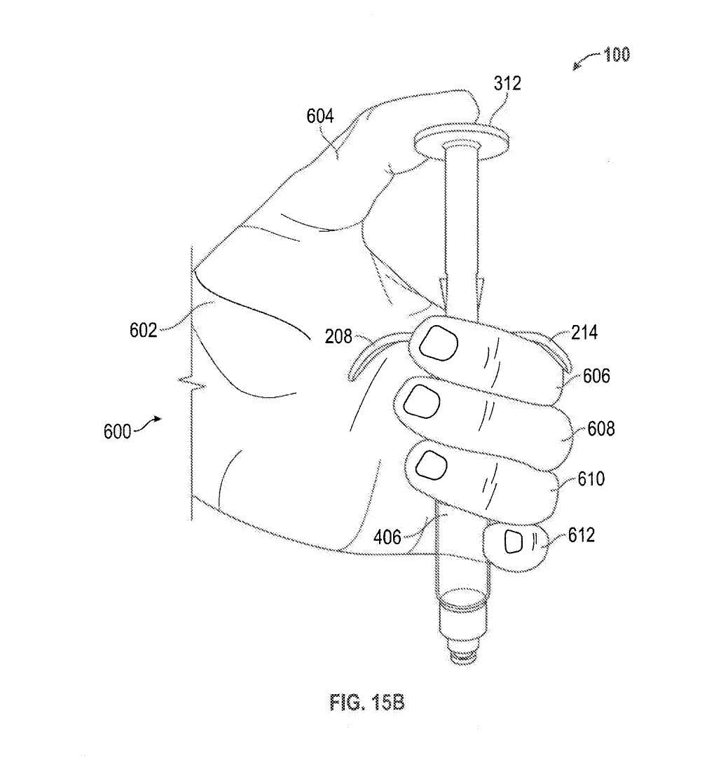

Turning now to FIGS. 15A, 15B, 16, 17A, and 17B, various embodiments of the syringe 100 according to the present invention are illustrated along with a patient or caregiver's fingers, thumb, and/or gripping, steadying, or otherwise manipulating the syringe 100 when utilizing the syringe 100. As explained above, because of one or more of the specific syringe 100 configurations outlined above, syringes 100 according to embodiments of the present invention may provide improved ergonomics, versatility, and/or ease of use to a user.

FIGS. 15A and 15B are illustrations of a user's hand 600 holding the syringe 100 according to an embodiment of the present invention. The style of grip illustrated in these figures may be referred to as the “joystick” grip. The joystick grip may be characterized by the user placing their index 606, middle 608, ring 610, and little 612 fingers beside the main body 406 of the barrel 400 of the syringe 100, as shown in FIG. 15A, and then wrapping these fingers around the main body 406 of the barrel 400, as shown in FIG. 15B. In each of these figures, one of the finger flanges of the finger flange assembly 200 (in this case the second flange 214) curves and wraps around a portion of the user's index finger 606.

In the illustrated embodiment, the other finger flange (in this case the first flange 208) may be secured in the palm 602 of the user's hand 600. In alternate embodiments, if the user so desires and if the syringe 100 so fits their hand 600, the first flange 208 may curve and wrap over the back side of the user's hand 600. In the embodiment of FIGS. 15A and 15B, the user's thumb 604 is free to move about and manipulate the head 312 of the plunger 300 to, for example, drive the plunger 300 distally downward to cause the material held by the barrel 400 to be expelled, or pull the plunger 300 proximally upward to draw material into the barrel 400.

Because of the configuration of the depicted embodiment of the syringe 100, the user can comfortably, ergonomically grasp the syringe 100 and may easily adjust their grip as necessary during an injection. In a typical intravenous injection for a hemophilia patient, the user is primarily depressing the plunger 300 to inject the medicament 418, but may also need to frequently draw back on the plunger 300 to look for blood in the flexible tube 508 of the infusion set 500 to ensure that the injection is in fact proceeding into a vein. Because of this, the user may need to occasionally move their fingers or thumb to switch between injection and draw-back positions, or may need to switch their style of grip entirely.

Compared to conventional syringe finger flange assemblies, which have more traditional finger flange and plunger configurations, embodiments of the present invention described above with respect to one or more of FIGS. 1-14 have finger flange assemblies 200 and plungers 300 allowing for improved ergonomics, versatility, and/or ease of use, particularly for a patient or caregiver administering an injection to a hemophilia patient.

For example, with respect to the joystick grip depicted in FIGS. 15A and 15B, an appropriately configured, curved, and tapered first flange 208 and second flange 214 may provide a hemophilia patient self-administering an injection with an ergonomic structure that is easy to use and grip during an injection. The first flange 208 and the second flange 214 are configured to prevent the user's fingers from unexpectedly slipping off the syringe 100 in the joystick grip, but still allow the user to withdraw their fingers with relative ease to establish a different grip for a different point of the injection cycle (e.g. draw-back). The finger flange assembly 200 as a whole is also suitably flexible in that the user may cradle one of the flanges in the palm of their hand, if desired, or allow the flange to cross over the back of their hand, if desired. Thus, in some embodiments, the configuration of the first flange 208 and the second flange 214 is such that it minimizes potential stress points while maximizing ergonomics and flexibility.

In addition, the head 312 of the plunger 300 including the dimple 318 are configured so that a user's thumb in the joystick grip may be able to easily locate and securely grip the head 312 of the plunger 300, and easily actuate the head 312 in both injection and draw-back phases. Again, in such an embodiment, the configuration of the head 312 of the plunger 300 is such that it minimizes potential stress points while maximizing ergonomics and flexibility.

Repeated unnecessary realignments and re-grips or undesirable ensnaring of a user's fingers or thumb can lead to overall poor ergonomics and the generation of microbleeds. As described above, this repeated microbleed generation over time has the potential to cause severe damage to the body of a person affected by hemophilia. Given the need for regularly scheduled blood clotting factor protein injections for many hemophilia patients, an optimally designed syringe offering improved ergonomics, versatility, and/or ease of use is of great value.

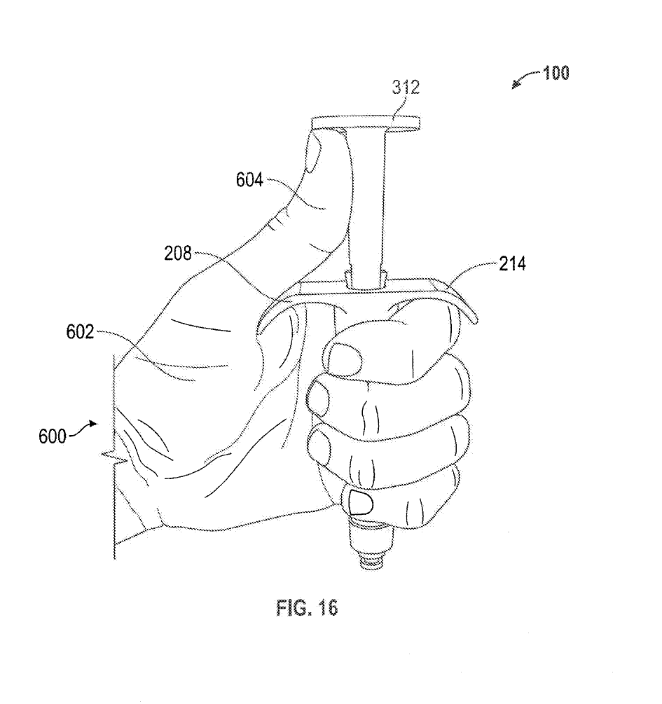

FIG. 16 is an illustration of a user's hand 600 holding the syringe 100 according to another embodiment of the present invention. Similar to FIGS. 15A and 15B, FIG. 16 illustrates the use of the joystick grip. However, in FIG. 16, the user's thumb 604 is positioned to draw back on the plunger 300. As described above, this may be desirable for the user to be able to look for blood in the flexible tube 508 of the infusion set 500 to ensure that the injection is in fact proceeding into a vein. Of course, in alternate embodiments, the user's thumb 604 or other portions of the user's hand may be positioned differently to achieve a draw back movement.

As explained in further detail above, drawback in useful in intravenous injections, such as, for example, injections to hemophilia patients, so that the user of the syringe 100 may confirm that the injection is in fact properly entering into a vein. Proper injection is confirmed when blood from the vein is drawn back into the syringe 100 temporarily. Compared to conventional syringes 100, which may have heads 312 of plungers 300 that are proportioned differently than those specified above, embodiments of the present invention with sufficiently large circular plunger 300 heads 312 may allow a user of the syringe 100 to more easily execute a draw back movement with their thumb in a way that does not require repeated realignments or manipulation struggles, which could lead to the generation of microbleeds.

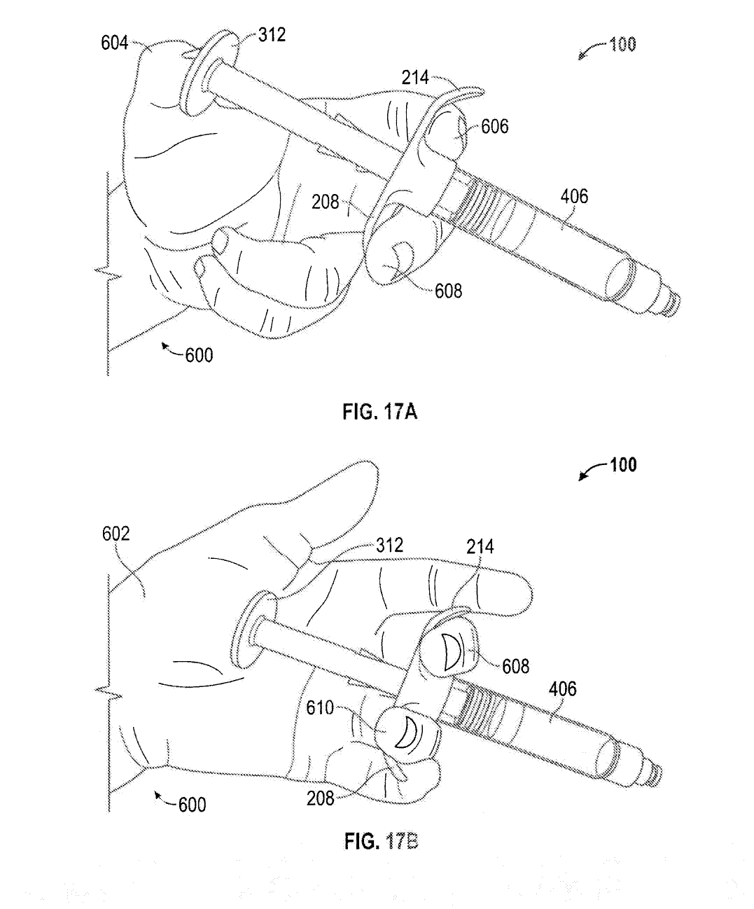

FIG. 17A is an illustration of a user's hand 600 holding the syringe 100 according to another embodiment of the present invention. The style of grip illustrated in these figures may be referred to as the “two-finger” grip. The two-finger grip may be characterized by the user placing their index finger 606 in one of the finger flanges of the finger flange assembly 200 (in this case the second flange 214), while placing their middle finger 608 in the other finger flange (in this case the first flange 208). In this embodiment, the first flange 208 and the second flange 214 each curve distally and wrap around portions of the user's middle 608 and index 606 fingers, respectively.

In alternate embodiments, if the user so desires and if the syringe 100 so fits their hand 600, the user may insert other fingers besides the middle 608 and index 606 fingers into the first flange 208 and the second flange 214. In the embodiment of FIG. 17A, the user's thumb 604 is free to move about and manipulate the head 312 of the plunger 300 to, for example, drive the plunger 300 distally downward to cause the material held by the barrel 400 to be expelled, or pull the plunger 300 proximally upward to draw material into the barrel 400.

The configuration of this exemplary syringe 100 offers some of the same benefits to a user employing that two-finger grip that it does to a user employing the joystick grip. For example, with respect to the two-finger grip depicted in FIG. 17A, an appropriately configured, curved, and tapered first flange 208 and second flange 214 may provide a hemophilia patient self-administering an injection with an ergonomic structure that is easy to use and grip during an injection. The first flange 208 and the second flange 214 are configured to prevent the user's fingers from unexpectedly slipping off the syringe 100 in the joystick grip, but still allow the user to withdraw their fingers with relative ease to establish a different grip for a different point of the injection cycle (e.g. draw-back). Note that in the two-finger grip of FIG. 17A, it is the user's fingertips that contact the finger flange assembly 200, not the side of the fingers as depicted in FIGS. 15A and 15B for the joystick grip. As with the joystick grip, even in the two-finger grip, the exemplary configuration of the finger flange assembly 200 is such that it minimizes potential stress points while maximizing ergonomics and flexibility.

FIG. 17B is an illustration of a user's hand 600 holding the syringe 100 according to another embodiment of the present invention. The style of grip illustrated in these figures may be referred to as the “palm press” grip. The palm press grip may be characterized by the user placing their middle finger 608 in one of the finger flanges of the finger flange assembly 200 (in this case the second flange 214), while placing their ring finger 610 in the other finger flange (in this case the first flange 208). In this embodiment, the first flange 208 and the second flange 214 each curve distally and wrap around portions of the user's ring 610 and middle 608 fingers, respectively.

In alternate embodiments, if the user so desires and if the syringe 100 so fits their hand 600, the user may insert other fingers besides the ring 610 and middle 608 fingers into the first flange 208 and the second flange 214. In the embodiment of FIG. 17B, it is the palm 602 of the user's hand 600 that is used to manipulate the head 312 of the plunger 300 to, for example, drive the plunger 300 distally downward to cause the material held by the barrel 400 to be expelled.

Compared to conventional syringe plunger heads, which typically have smaller plunger head surface areas and no dimples, embodiments of the present invention described above with respect to one or more of FIGS. 1-14 and depicted in FIG. 17B may have a plunger head 312 with a spherical dimple 318 that may allow a user to easily locate and securely grip the head 312 of the plunger 300, and easily actuate the head 312. Again, in such an embodiment, the configuration of the head 312 of the plunger 300 is such that it minimizes potential stress points while maximizing ergonomics and flexibility.

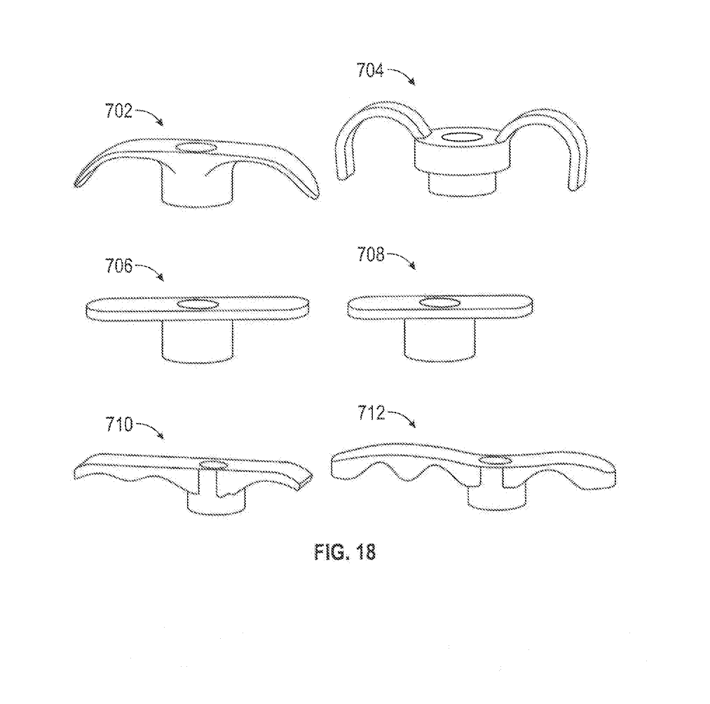

FIG. 18 illustrates various alternative finger flange assemblies 200 according to embodiments of the present invention. In one embodiment, the finger flange assembly 702 may include flanges with a relatively gradual taper as compared to the first flange 208 and second flange 214 shown in FIGS. 7A, 7B, 8A, and 8B. In another embodiment, the finger flange assembly 704 may include semicircular shaped flanges. In further embodiments, the finger flange assembly 706 may include relatively long, flat flanges, while the finger flange assembly 708 may include relatively short flat flanges. Finally, in still other embodiments, the finger flange assembly 710 may include relatively shallow ridges for cradling three or more individual fingers of a user's hand, while the finger flange assembly 712 may include relatively deep ridges for cradling three or more individual fingers of a user's hand. Finger flange assemblies 200 having additional configurations may be within the scope of embodiments of the present invention.

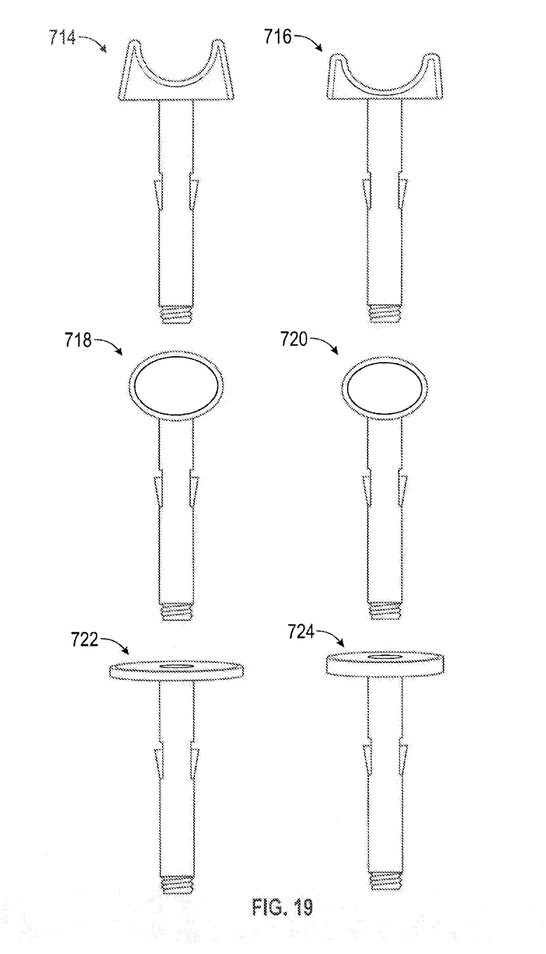

FIG. 19 illustrates various alternative plungers 300 according to embodiments of the present invention. In some embodiments, the plunger 714 may include a relatively deep upward-facing thumb cradle, while the plunger 716 may include a relatively shallow upward-facing thumb cradle. In other embodiments, the plunger 718 may include a relatively large circular thumb ring, while the plunger 720 may include a relatively small circular thumb ring. Finally, in further embodiments, the plunger 722 may include a relatively wide but shallow plunger head as compared to the head 312 of the plunger 300 shown in FIGS. 7A, 7B, 8A, and 8B, while the plunger 724 may include a relatively deep but narrow plunger head as compared to the head 312 of the plunger 300 shown in FIGS. 7A, 7B, 8A, and 8B. Plungers 300 having additional configurations may be within the scope of embodiments of the present invention.

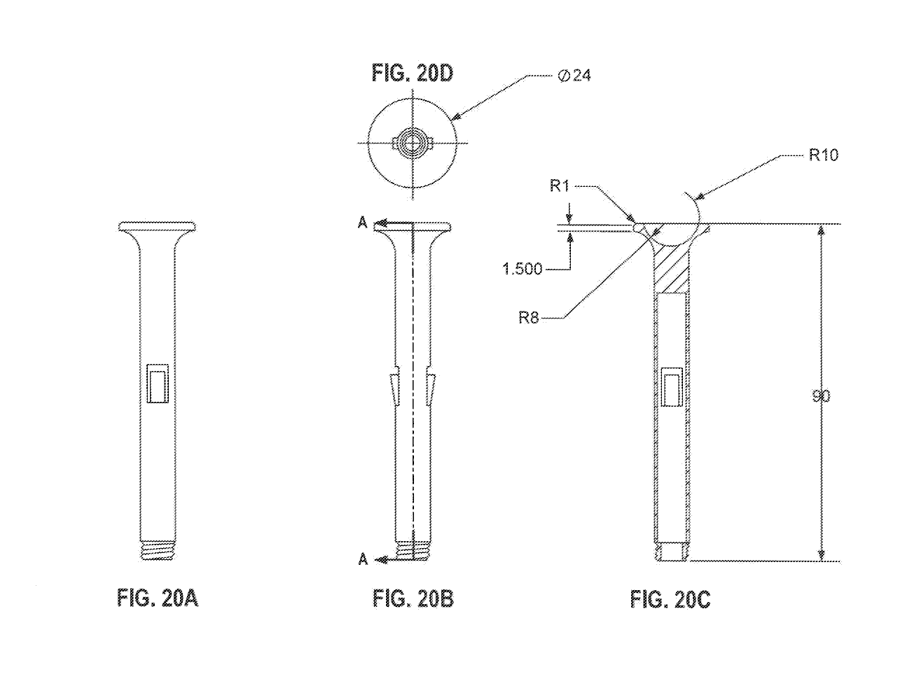

FIGS. 20A-20D are various schematic views of an alternative plunger 300 according to an embodiment of the present invention. Exemplary measurements of portions of the plunger 300 of this embodiment, which may differ in some respects from measurements of portions of the plunger 300 of the embodiment of FIGS. 14A-14D, are provided.

In one embodiment of the present invention, parts of the syringe 100 may include portions with increased texture or a different material to aid in the ability of a user to grip the syringe 100. For example, the first flange 208 and the second flange 214 may include first and second rubber portions, respectively, on their proximal and/or distal surfaces. In another embodiment, the head 312 of the plunger 300 may include a rubber portion on its proximal and/or distal surface. In other embodiments, increased texture maybe provided by using materials other than rubber or by scoring, stippling, dimples, ridges, or other textural surface elements.

In a further embodiment of the present invention, different syringes 100 may be designed for different doses of medicament 418. For example, syringes 100 may be pre-filled with medicaments 418 having different concentrations, volumes, and/or strengths. In an embodiment, a portion of each syringe 100 may be colored with a specific color, wherein the syringe 100 is color coded to indicate the dose of the medicament 418 that it contains. For example, the texturized or rubberized portions of the syringe 100 described above may be color coded.

In another embodiment of the present invention, parts of the syringe 100 may include portions having product labeling. Labeling may be included by the addition of label stickers or by having the labeling printed, caved, molded, or otherwise formed on or in parts of the syringe 100. In some embodiments the syringe 100 is labeled to indicate the dose of the medicament 418 that it contains.

In another aspect, the invention is directed to a kit comprising the syringe 100 described herein and a package insert. The package insert may include an instruction for using the syringe 100, for administering the medicament 418, or for treating a disorder or condition. The kit may comprise a medicament 418, which may be contained in the syringe 100 or may be contained separately from the syringe 100. The medicament 418 may be in unit dosage form in one of the doses described herein.

It should be noted, that while the above discussion focuses primarily on the use of syringes for medical purposes generally, and for the treatment of hemophilia specifically, the embodiments of delivery devices described herein need not be limited to syringes, need not be so limited in application, and may be suitable for a variety of non-medical purposes, or for treating medical conditions beyond hemophilia.

The present invention has been described above by way of exemplary embodiments. Accordingly, the present invention should not be limited by any of the above-described exemplary embodiments, but should be defined only in accordance with the following claims and their equivalences.

What is claimed is:

1. A delivery device comprising:
a barrel holding a liquid for delivery;
a finger flange assembly coupled to said barrel, said finger flange assembly comprising:
a first flange and a second flange each extending laterally from opposite sides of said finger flange assembly, wherein the first flange and the second flange each curve toward said barrel; and
a plunger coupled to said barrel and passing through said finger flange assembly, said plunger comprising a disc-shaped head.

2. The delivery device of claim 1, wherein the delivery device comprises a syringe.

3. The delivery device of claims 1 and 2, wherein said barrel is configured to hold a diluent and a medicament in isolation from one another.

4. The delivery device of claim 1 to 3, said barrel further comprising:
a main body of the barrel configured to hold a diluent and a medicament in isolation from one another, wherein the main body includes a bypass; and
a moveable piston coupled to the main body,
wherein the piston and bypass are configured such that relative movement of the piston with respect to the bypass can result in contact between the diluent and the medicament.

5. The delivery device of any one of claims 1 to 4,
wherein the first flange and the second flange each terminate in respective first and second flange tips, and
wherein the first flange and the second flange each become narrower as they extend toward their respective first and second flange tips.

6. The delivery device of claim 5,
wherein the delivery device defines a central axis running down the center of the delivery device in the proximal-distal direction, and
wherein the distance between the first flange tip and the proximal-most extent of said finger flange assembly, as measured parallel to the central axis, is equal to the distance between the second flange tip and the proximal-most extent of said finger flange assembly, as measured parallel to the central axis, and
wherein the distance is between 10 mm and 15 mm.

7. The delivery device of claims 5 and 6,
wherein the delivery device defines a central axis running down the center of the delivery device in the proximal-distal direction, and
wherein the distance between the first flange tip and the second flange tip, as measured perpendicular to the central axis, is between 62 mm and 67 mm.

8. The delivery device of claim 5 to 7,
wherein the delivery device defines a central axis running down the center of the delivery device in the proximal-distal direction,
wherein the distance between the first flange tip and the proximal-most extent of said finger flange assembly, as measured parallel to the central axis, is equal to the distance between the second flange tip and the proximal-most extent of said finger flange assembly, as measured parallel to the central axis, and the distance is defined as X,
wherein the distance between the first flange tip and the second flange tip, as measured perpendicular to the central axis, is defined as Y, and
wherein the ratio of X to Y is between 1:4 and 1:6.

9. The delivery device of any one of claims 1 to 8, wherein the spherical dimple is centered on the proximal surface of the plunger head.

10. The delivery device of any one of claims 1 to 9, wherein the proximal surface of the plunger head includes only a single spherical dimple, and wherein the remainder of the proximal surface of the head is flat.

11. The delivery device of any one of claims 1 to 10, wherein the plunger head has a diameter of greater than 25 mm at its widest portion.

12. The delivery device of any one of claims 1 to 11, wherein the ratio of the diameter of the plunger head at its widest portion to the dimple diameter is between 2:1 and 3:1, wherein the dimple diameter is defined as the diameter of the spherical dimple as measured at the proximal surface of the head.

13. The delivery device of any one of claims 1 to 11, wherein the ratio of the diameter of the plunger head at its widest portion to the dimple diameter is between 1.0:1 and 1.5:1, wherein the dimple diameter is defined as the diameter of the spherical dimple as measured at the proximal surface of the head.

14. The delivery device of any one of claims 1 to 13, said plunger further comprising:
a rod coupled to and extending distally from the plunger head, wherein the rod passes through said finger flange assembly.

15. The delivery device of claim 14, wherein the external boundary between the plunger head and the rod is defined by a circular incurvate edge.

16. The delivery device of claim 15, wherein the incurvate edge has a radius of curvature of between 1 mm and 3 mm.

17. The delivery device of claim 15, wherein the incurvate edge has a radius of curvature of between 6 mm and 10 mm.

18. The delivery device any one of claims 1 to 17, said barrel further comprising:
a main body configured to hold the liquid; and
a hub coupled to the main body configured to transfer the liquid to into an infusion set,
wherein the hub is configured so that a user visually inspecting the hub may discern whether substantially all of the liquid has been transferred out of the hub and into the infusion set.

19. The delivery device any one of claims 1 to 18, wherein the first flange and the second flange include first and second grip enhancing portions, respectively, on their distal surfaces.

20. The delivery device of claim 19, wherein the plunger head includes a grip enhancing portion on its proximal surface.

21. The delivery device of any one of claims 1 to 20, wherein the syringe is color coded to indicate the dose of a medicament that it contains.

22. A finger flange assembly configured for use with a delivery device, the finger flange assembly comprising:
a substantially cylindrical trunk having a proximal end and a distal end, wherein the distal end of said trunk is configured to attach to the barrel of the delivery device; and
a first flange and a second flange each extending laterally from opposite sides of the proximal end of said trunk, wherein said first flange and said second flange each curve toward the distal direction and terminate in respective first and second flange tips, wherein said first flange and said second flange each become narrower as they extend toward the respective first and second flange tips.

23. The finger flange assembly of claim 22 further comprising:
an aperture in said trunk running in the proximal-distal direction.

24. The finger flange assembly of claim 23, wherein the aperture comprises:
a circular proximal aperture opening on the proximal end of said trunk; and
a circular distal aperture opening on the distal end of said trunk,
wherein the inner diameter of the circular proximal aperture opening is less than the inner diameter of the circular distal aperture opening.

25. The finger flange assembly of any one of claims 22 to 24,
wherein said trunk defines a central axis running down the center of said trunk in the proximal-distal direction, and
wherein the first and second flange tips are equidistant from the central axis.

26. The finger flange assembly of any one of claims 22 to 25,
wherein said trunk defines a central axis running down the center of said trunk in the proximal-distal direction, and
wherein the distance between the first flange tip and the proximal-most extent of the finger flange assembly, as measured parallel to the central axis, is equal to the distance between the second flange tip and the proximal-most extent of the finger flange assembly, as measured parallel to the central axis, wherein the distance is defined as X.

27. The finger flange assembly of claim 26, wherein X is between 10 mm and 15 mm.

28. The finger flange assembly of any one of claims 22 to 27,
wherein said trunk defines a central axis running down the center of said trunk in the proximal-distal direction, and
wherein the distance between the first flange tip and the second flange tip, as measured perpendicular to the central axis, is between 62 mm and 67 mm.

29. The finger flange assembly of any one of claims 22 to 27,
wherein said trunk defines a central axis running down the center of said trunk in the proximal-distal direction,
wherein the distance between the first flange tip and the proximal-most extent of the finger flange assembly, as measured parallel to the central axis, is equal to the distance between the second flange tip and the proximal-most extent of the finger flange assembly, as measured parallel to the central axis, wherein the distance is defined as X,
wherein the distance between the first flange tip and the second flange tip, as measured perpendicular to the central axis, is defined as Y, and
wherein the ratio of X to Y is between 1:4 and 1:6.

30. A plunger configured for use with a delivery device, the plunger comprising:
a circular disc-shaped head at a proximal end of the plunger; and
a rod coupled to and extending distally from said head,
wherein the proximal surface of said head includes a spherical dimple.

31. The plunger of claim 30, wherein the spherical dimple is centered on the proximal surface of said head.

32. The plunger of claims 30 or 31,
wherein the proximal surface of said head includes only a single spherical dimple, and
wherein the remainder of the proximal surface of said head is flat.

33. The plunger of any one of claims 30 to 32, wherein said head has a depth between 2 mm 3 mm.

34. The plunger of any one of claims 30 to 33, wherein said head has a diameter of greater than 25 mm at its widest portion.

35. The plunger of any one of claims 30 to 34, wherein the ratio of the diameter of said head at its widest portion to the dimple diameter is between 2:1 and 3:1, wherein the dimple diameter is defined as the diameter of the spherical dimple as measured at the proximal surface of said head.

36. The plunger of any one of claims 30 to 34, wherein the ratio of the diameter of said head at its widest portion to the dimple diameter is between 1.0:1 and 1.5:1, wherein the dimple diameter is defined as the diameter of the spherical dimple as measured at the proximal surface of said head.

37. The plunger of any one of claims 30 to 36, wherein the spherical dimple has a radius of curvature of between 7.5 mm and 12.5 mm.

38. The plunger of any one of claims 30 to 37, wherein the external boundary between said head and said rod is defined by a circular incurvate edge.

39. The plunger of claim 38, wherein the incurvate edge has a radius of curvature of between 1 mm and 3 mm.

40. The plunger of claim 38, wherein the incurvate edge has a radius of curvature of between 6 mm and 10 mm.

41. The plunger of claim 38, wherein the ratio of the diameter of said head at its widest portion to the diameter of said rod at a level just distal to the incurvate edge is greater than 2.5:1.

42. A delivery device comprising:
a barrel holding a liquid for delivery;
the finger flange assembly of any one of claims 22 to 29, wherein the finger flange assembly is coupled to the barrel; and
the plunger of any one of claims 30 to 41, wherein the plunger is coupled to the barrel and passes through the finger flange assembly.

43. The delivery device of any one of claims 1 to 21 and 42, wherein the liquid comprises a medicament.

44. The delivery device of any one of claims 1 to 21 and 43, wherein the medicament comprises a protein capable of treating a blood clotting disorder.

45. The delivery device of claim 44, wherein the protein is a Factor VIII protein.

46. The delivery device of any one of claim 44, wherein the protein is a Factor IX protein.

47. A kit comprising the delivery device of any one of claims 1 to 21 and 42 to 46 and a package insert.

48. The kit of claim 47, wherein the package insert includes instructions for using the delivery device.

49. The delivery device of claim 4, wherein the main body of the barrel is configured to allow for drawback during an injection.

50. The delivery device of claim 49, wherein the main body of the barrel is configured to allow for sufficient drawback during an injection such that a user of the delivery device can determine that an injection is in fact properly entering into a vein.

51. The delivery device of claims 49 and 50, wherein the main body has a total volume of approximately 2.5 ml distal to the bypass.

52. The delivery device of claim 49 to 51, wherein the main body has a length of approximately 45 mm distal to the bypass 408.
